(19) United States
(12) Patent Application Publication
(10) Pub. No.: US 2025/0381446 A1
(43) Pub. Date: Dec. 18, 2025

(54) SYSTEM, DEVICE AND METHOD FOR HAND THERAPY WITH ACCURATE FORCE MEASUREMENT

(71) Applicant: MINDMAZE GROUP SA, Lausanne (CH)

(72) Inventors: Nicholas FREMAUX, Lausanne (CH); Sylvain CARDIN, Lausanne (CH); Mouna CHERAKA, Lausanne (CH); Gangadhar GARIPELLI, Lausanne (CH); Naveed EJAZ, Lausanne (CH); Tamarah SUYS, Lausanne (CH); Calan APPADU, Lausanne (CH); Gaëlle MINGER, Lausanne (CH); Cyntia PARENT, Lausanne (CH); Sylvain TRAEGER, Lausanne (CH); Helder RODRIGUE, Lausanne (CH)

(21) Appl. No.: 19/062,113

(22) Filed: Feb. 25, 2025

Related U.S. Application Data
(63) Continuation of application No. PCT/IB2023/058410, filed on Aug. 24, 2023.
(60) Provisional application No. 63/401,120, filed on Aug. 25, 2022.

(51) Int. Cl. A63B 24/00; A63B 21/00; A63B 23/16

(52) U.S. Cl. CPC A63B 24/0062; A63B 21/4023; A63B 23/16; A63B 2024/0065; A63B 2220/40; A63B 2220/56; A63B 2220/58; A63B 2220/74; A63B 2225/02; A63B 2225/50

(57) ABSTRACT

Devices, methods and systems related to the assessment and therapy of neurological conditions and dexterous hand function in particular. For example, some embodiments can relate to devices comprising a first portion of flexible material and forming a cavity; a second portion non-flexible material; a PCB coupled to the second portion; and wherein a portion of an edge of the first portion is configured to create a semi-hermetic seal with at least a portion of an edge of the second portion and the PCB has connected thereto a pressure sensor, a wireless transceiver, and a power storage unit.

### FIELD OF THE DISCLOSURE

The present disclosure relates to devices, systems, and methods for the assessment and therapy of neurological conditions and conditions affecting dexterous hand function in particular.

### BACKGROUND

For clinical practice, professional associations provide recommendations about which scales to use like the Academy of Neurological Physical Therapy (ANPT) in the United States. These recommendations are usually country and condition specific. It is up to each site to implement them in current practice.

For clinical research, the Stroke Recovery and Rehabilitation Roundtable (SRRR) have published this year guidelines about how to measure quality of movement for upper limb in patients suffering from stroke (Kwakkel et al. 2019).

In current practice, each clinic is using its own set of scales in order to assess patients' impairments.

Typically, impairments are assessed in isolation, it is rare to have a full battery of assessments that include upper limbs, lower limbs, hand and cognition. Performing the assessment is time consuming and gives you a score with different degrees of sensitivity and variability. An automated multi-modal assessment would give the best fidelity in tracking impairments and providing a holistic view of patients' impairments.

Furthermore, a major challenge in neurorehabilitation is that recovery can either be behavioral restitution or behavioral compensation. It is critical to measure to deliver the most adequate therapy to each patient. The automated multi-modal assessment could potentially overcome this challenge while removing inter-rater variability and saving time by integrating all dimensions in a gamified activity that is part of the patients' therapy treatment plan.

Physical and occupational therapists create a tailored therapy plan for each patient based on the patient's medical record and their examination of the patient's nature of impairment and the associated severity. The therapy plan includes the type of therapy, the dosage as well as the schedule (i.e. how often the therapy sessions will take place). Finding the appropriate therapy plan is complicated without a holistic view of the patient's sensorimotor and cognitive impairments. Each therapist develops his/her own protocols to treat patients depending on if he is a physical therapist, an occupational therapist or a neuropsychologist. This lack of homogeneity prevents the field from identifying the optimal therapy treatment according to the patient's level of impairment.

Moreover, these problems are magnified in cases where treatment options are limited or fail to target specific areas in need of either assessment or therapy. One such area is hand function. Humans have a unique ability to use their hands in a dexterous manner to manipulate and interact with the environment around them. Dexterous hand function in humans is primarily controlled by the corticospinal pathway. The corticospinal pathway can be damaged because of neurological insult (e.g., stroke, spinal cord injury). Since the corticospinal pathway is the primary pathway that controls hand function, dexterous hand function is impaired in patients suffering from a neurological insult.

In neurorehabilitation, the current focus in hand training is mostly corrupted by strength requirements of the task. Consequently, most clinical instruments and devices primarily measure deficits in strength. There is currently no valuable clinical instrument nor device to measure deficits in dexterity. After brain injury, there are some stereotypical deficits that emerge:
Loss of hand control masked by synergies.
Inability to fully open the hand due to problems focally engaging extensor muscles.
Impaired ability to perform functional tasks (e.g., grabbing and stabilizing a cup).

Current devices and methods fail to take into account these deficits without measuring and treating strength either as a primary function or because of inherent bias. For example, dynamometers and other devices and their methods that measure force exhibit this bias towards training strength.

In neurorehabilitation, even clinical instruments and methods that are intended to measure dexterity fail to isolate dexterity properly. Such methods include the Box and Block Test and the Nine Whole Peg Test. Such devices and methods additionally cannot be used with patients that have severe dexterity deficits. That is, a minimum dexterity is needed to be able to execute the tasks included in these instruments. It is uncertain if improvement in dexterity as measured with these instruments is really reflecting improvement in dexterity rather than functional improvement related to the learning phase of the tasks included in these instruments.

Other existing technologies meant to train dexterity deficits (e.g., Gloreha Sinfonia, MusicGlove, HandTutor and Neofect) use intensity and dosage as measures for dexterity, which is not sufficient as a measure.

Thus, what is needed is a device and methods for integrating a logic targeting the training of these impairments/functions and that are sensitive enough to measure dexterity deficits objectively.

### SUMMARY OF SOME OF THE EMBODIMENTS

Therapists can use various embodiments of the devices and methods described herein for whole hand grasp training, pincer grasp training, as well as in other configurations. Such devices and methods can be used in different postures allowing spastic patients to train with them.

Some embodiments of the present disclosure are peripheral devices that can connect via Bluetooth and allow game interactions by providing information about grasp force and orientation. Some embodiments are egg-shaped devices that the user holds in the hand and a base station for charging and pressure resetting.

According to at least some embodiments, there is provided a device for providing dexterous hand function assessment and therapy, comprising: a first portion of flexible material forming a cavity: a second portion of non-flexible material: a plurality of electronics coupled to the second portion: and wherein a portion of an edge of the first portion is configured to create a partial seal with at least a portion of an edge of the second portion, such that said cavity is partially sealed: said plurality of electronics comprises a pressure sensor, a wireless transceiver, and a power storage unit: said electronics are located within said cavity: and said partial seal is non-hermetic. Optionally, the first portion comprises a graphical component to enable position and orientation tracking of the device. Optionally, the device further comprises an active marker to enable position and orientation tracking of the device. Optionally, the device further comprises a third portion through which the active marker is visible.

Optionally, said electronics further comprise one or more inertial measurement units (IMU) and a memory storage device. Optionally, said pressure sensor comprises a barometric pressure sensor within said cavity for measuring pressure within said cavity. Optionally, the device further comprises a temperature sensor. Optionally, temperature data from said temperature sensor and pressure data from said pressure sensor are transmitted from the device through said wireless transceiver for determination of an external force applied to said first portion of flexible material. Optionally, said electronics are anchored to a PCB board. Optionally, said first portion of flexible material comprises a material selected from the group consisting of silicone and another flexible polymer. Optionally, said second portion of non-flexible material comprises a sufficiently flat facet such that the device can rest upon said facet: and said non-flexible material of said second portion does not deform under the weight of the device when the device is resting on said facet. Optionally, said first portion of flexible material is adapted to be manipulated by a hand to receive applied force. Optionally, measurements of said applied force are not captured through said second portion. Optionally, said electronics are anchored to said second portion within said cavity, through a stationary and non-deformable anchor point in the second portion. Optionally, said electronics are anchored to said second portion through a PCB.

Optionally, the device further comprises a valve to allow the air pressure on the interior of the device to adjust to equilibrium with ambient air pressure. Optionally, said valve is located in said second portion. Optionally, said valve is located in said first portion. Optionally, the device further comprises a pressor surface for being pressed for activating said valve. Optionally, the device further comprises a reset button for resetting said electronics, said reset button comprising said pressor surface. Optionally, said valve is activated to equalize internal pressure of the device with ambient pressure.

Optionally, the device further comprises a reset button for resetting said electronics. Optionally, said wireless transceiver uses at least one of a Bluetooth, cellular data, or RFID wireless system.

According to at least some embodiments, there is provided a system for providing dexterous hand function assessment and therapy, comprising: the device as described herein: and a base station comprising a battery charging component, the battery charging component including a cooling system. Optionally, the system further comprises an external computational device: wherein the pressure within the device is determined according to said pressure sensor data and said temperature sensor data from said temperature sensor within said cavity: and wherein an applied force to the device is determined by said external computational device according to the pressure and the temperature data. Optionally, calibration data is received by said external computational device, said calibration data comprising a first pressure measurement of the interior of the device: wherein said first pressure measurement is a baseline. Optionally, said first pressure measurement comprises a barometric pressure measurement. Optionally, a calibration process is performed with a user manipulating the device, wherein said calibration process further comprises performing a plurality of manipulations of the device, comprising a movement of the device and a grip on the device, to determine at least one of user range of motion, a maximum pressure for gestures, a minimum acceleration, a maximum acceleration, or a combination thereof. Optionally, said user range of motion is determined from accelerometer data. Optionally, said user range of motion comprises wrist flexion/extension, and wherein a range of motion of wrist flexion/extension is determined by deriving a first plane from an acceleration vector when the device is held by the user at maximum wrist flexion, and by deriving a second plane when the device is held by the user at maximum wrist extension: wherein said range of motion is determined according to an angle between these two vectors on the plane.

Optionally, said wireless transceiver uses at least one of a Bluetooth, cellular data, or RFID wireless system. Optionally, the device comprises a processor for operating firmware, wherein said firmware controls a connection between the device and said external computational device, for streaming data from the device to said external computational device. Optionally, said streaming data is streamed as packets, according to one or more streaming data parameters to select and configure packet content, comprising one or more of timestamp data, acceleration data, gyroscope data, pressure data, temperature data, data normalized according to calibration, or gyroscope derived quaternion data, or a combination thereof. Optionally, said accelerometer data is processed through a signal processing to dampen a signal from said accelerometer with minimal impact on latency for use during calibration and activity use.

According to at least some embodiments, there is provided a method for the assessment and therapy of dexterous hand function, comprising: establishing a connection with a hand device in a system as described herein: establishing a hand selection assigned to the hand device: receiving a first pressure measurement of the interior of the hand device: and sending an instruction to an idle pressure controller indicating the pressure measurement is a baseline. Optionally, said pressure measurement comprises barometric pressure.

Optionally, the method further comprises receiving temperature data from the hand device: and determining an external force applied to the hand device at said first portion to at least the first portion according to the temperature data and the pressure data. Optionally, said determining the applied external force is determined according to a trained model. Optionally, the method further comprises performing a calibration process with a user manipulating the device, comprising performing a plurality of manipulations of the device, comprising a movement of the device and a grip on the device, to determine at least one of user range of motion, a maximum pressure for gestures, a minimum acceleration, a maximum acceleration, or a combination thereof.

Advantages of embodiments of the present disclosure include allowing the objective measurement of dexterity deficits and training dexterity deficit using the same hand device.

Unless otherwise defined, all technical and scientific terms used herein have the same meaning as commonly understood by one of ordinary skill in the art to which this disclosure belongs. The materials, methods, and examples provided herein are illustrative only and not intended to be limiting.

Various embodiments of the methods, systems and apparatuses of the present disclosure can be implemented by hardware and/or by software or a combination thereof. For example, as hardware, selected steps of methodology according to some embodiments can be implemented as a chip and/or a circuit. As software, selected steps of the methodology (e.g., according to some embodiments of the disclosure) can be implemented as a plurality of software instructions being executed by a computer (e.g., using any suitable operating system). Accordingly, in some embodiments, selected steps of methods, systems and/or apparatuses of the present disclosure can be performed by a processor (e.g., executing an application and/or a plurality of instructions).

Although embodiments of the present disclosure are described with regard to a “computer,” and/or with respect to a “computer network,” it should be noted that optionally any device featuring a processor and the ability to execute one or more instructions is within the scope of the disclosure, such as may be referred to herein as simply a computer or a computational device and which includes (but not limited to) any type of personal computer (PC), a server, a cellular telephone, an IP telephone, a smartphone or other type of mobile computational device, a PDA (personal digital assistant), a thin client, a smartwatch, head mounted display or other wearable that is able to communicate wired or wirelessly with a local or remote device. To this end, any two or more of such devices in communication with each other may comprise a “computer network.”

### BRIEF DESCRIPTION OF THE DRAWINGS

Embodiments of the disclosure are herein described, by way of example only, with reference to the accompanying drawings. With specific reference now to the drawings in detail, it is stressed that particulars shown are by way of example and for purposes of illustrative discussion of the various embodiments of the present disclosure only and are presented in order to provide what is believed to be a useful and readily understood description of the principles and conceptual aspects of the various embodiments of inventions disclosed therein.

FIG. 1 illustrates a schematic of an exemplary hand device in accordance with embodiments.

FIG. 2 illustrates a block diagram of components of an exemplary hand device in accordance with embodiments.

FIG. 3 illustrates components of an exemplary hand device in accordance with embodiments.

FIG. 4 illustrates a cross-section of a portion of an exemplary hand device in accordance with embodiments.

FIG. 5 illustrates a schematic of movements measured by exemplary hand devices in accordance with embodiments.

FIGS. 6A, 6B and 7 illustrate a schematics of exemplary hand devices in accordance with embodiments.

FIG. 8 illustrates a block diagram of an exemplary architecture in accordance with embodiments.

FIGS. 9-13 illustrate flowcharts of exemplary methods of operation of a hand device in accordance with embodiments.

FIG. 14 illustrates a flowchart for an exemplary method of assessment and therapy in accordance with embodiments.

FIGS. 15A and 15B illustrate user interfaces of an exemplary system of assessment and therapy in accordance with embodiments.

FIG. 16 illustrates various impairment function vectors in accordance with embodiments.

FIG. 17 illustrates predefined digital therapies with scale values in accordance with embodiments.

FIG. 18 illustrates a catalog of activities in an exemplary system of assessment and therapy in accordance with embodiments.

FIG. 19 illustrates a therapy plan structure in an exemplary system of assessment and therapy in accordance with embodiments.

FIG. 20 illustrates a detailed view of a therapy plan structure in an exemplary system of assessment and therapy in accordance with embodiments.

FIG. 21 illustrates a user interface view of recommended digital therapy plans in an exemplary system of assessment and therapy in accordance with embodiments.

FIGS. 22 and 23 illustrate user interfaces for adjusting assessment and therapy parameters in an exemplary system of assessment and therapy in accordance with embodiments.

FIG. 24 illustrates a user interface for difficulty parameters in an exemplary system of assessment and therapy in accordance with embodiments.

FIG. 25 illustrates a user interface showing the relationship between game-states and corresponding kinematics in an exemplary system of assessment and therapy in accordance with embodiments.

FIG. 26 illustrates a user interface showing therapy progress in an exemplary system of assessment and therapy in accordance with embodiments.

FIG. 27 illustrates a flowchart of an exemplary method for closed-loop assessment and treatment of a neurological condition in accordance with embodiments.

FIGS. 28A and 28B illustrate block diagrams of components of an exemplary hand device in accordance with embodiments.

FIG. 29 shows a non-limiting, exemplary method for determining force applied to an exemplary hand device in accordance with embodiments.

FIG. 30 shows a non-limiting, exemplary method for training a model in regard to determining force applied to an exemplary hand device in accordance with embodiments.

FIGS. 31A-31B show different exemplary embodiments of the test robot system.

FIGS. 32A-32B show non-limiting examples of data reported by the test robot system.

FIG. 33 shows the measured relationship between temperature and pressure.

FIGS. 34A-C show the terms used in the models (FIG. 34A) and the results from the models as applied to pinch (34B) and grasp (34C) hand manipulation modalities.

FIG. 35A shows a cross-section of an additional embodiment of the hand-held device.

FIG. 35B shows an exploded view of the additional embodiment of the hand-held device of FIG. 35A.

### DETAILED DESCRIPTION OF SOME OF THE EMBODIMENTS

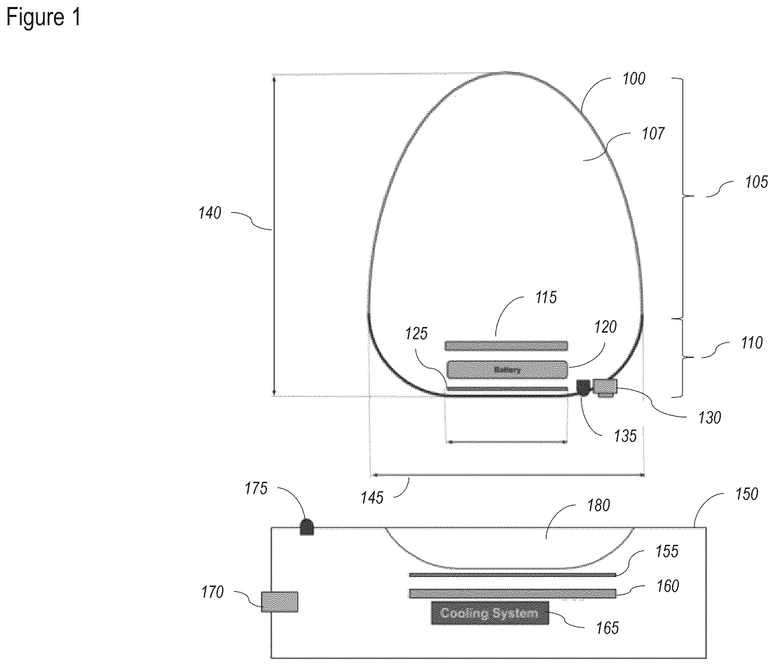

FIG. 1 illustrates an exemplary schematic of a device 100 in accordance with embodiments. Shown are two portions of the device 100 and a base 150. The top portion 105 is a compressible material preferably biocompatible so that the device can be used in sterile environments such as hospitals. Top portion 105 forms a cavity 107 on the interior of the device 100. In some embodiments, components of the device 100 describe further below can lie within the cavity 107. In other embodiments, components can lie only within a cavity of the bottom portion. The bottom portion 110 is a rigid material with a flat or sufficiently flat facet on its surface. By “sufficiently” flat, it is meant a facet that is level enough for the device to rest upon it. The bottom portion is preferably rigid to provide a stable area to mount the electronics and other internal components, to protect those components and to provide stable surface portion so it can stand upright when not in use or for mounting the device for charging or storage, for example, on the base 150 or a flat surface. Preferred embodiments of the device have an outer material that creates friction with the skin of the hand to reduce slipping from a user's hand. Preferably the flat surface is provided with a non-flexible material for stability. That is, the material of the portion of the device that includes the flat surface is preferably constructed from material that does not deform under the weight of the device when the device is resting on a surface such as a charging base. It is expected that the non-flexible portion is not a portion of the device through which force or interior pressure measurements are captured. A further advantage of a non-flexible portion is that assembly of the device is made simpler because electronics can be introduced to the interior of the device through a partition in the non-flexible portion, anchored to a substantially stationary and substantially non-deformable anchor point in the non-flexible portion. By “substantially stationary” it is meant that the anchor point does not move or may move only up to 10 millimeters. By “substantially non-deformable” it is meant that the anchor point does not deform or may deform only to the extent that the function as an anchor point is maintained. The non-flexible portion may comprise a bio-compatible hardened plastic or other polymer. In preferred embodiments the anchor point is contiguous with or of the same material as the non-flexible portion.

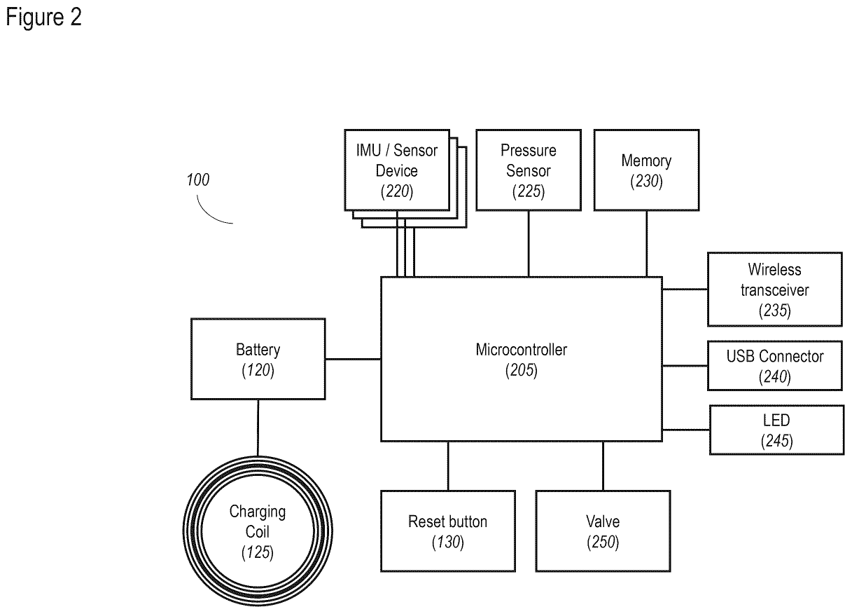

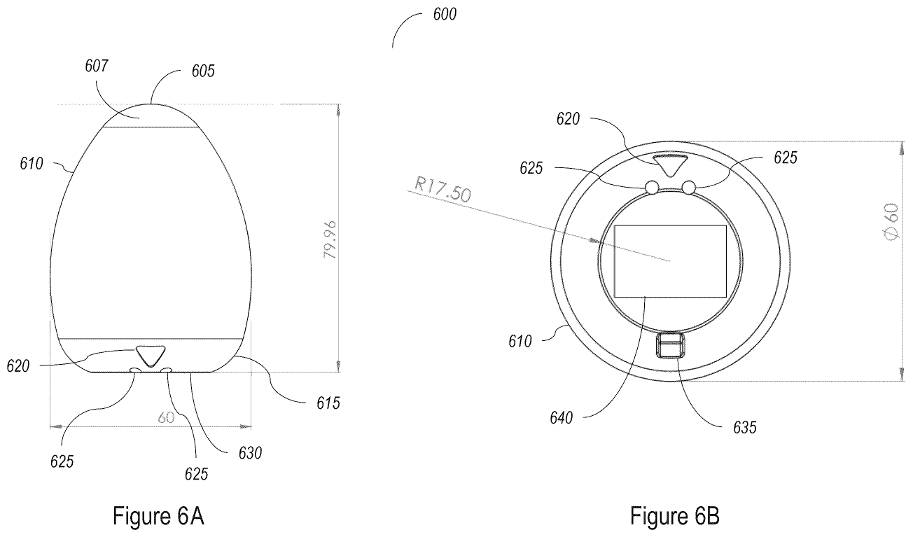

Device 100 includes a PCB 115 for the various components and a microcontroller, discussed further below. The bottom portion 110 preferably includes a reset button 130 and LED 135. Reset button 130 can be used to reset or calibrate the one or more sensors, particularly a barometer or other pressure sensor, which are discussed further below. Prior art devices are airtight so that air pressure on the interior of the device can be properly maintained. The inventors have found, however, that designing, manufacturing and maintaining a device that need not be hermetically sealed is simpler and more cost-effective. Accordingly, reset button 130 preferably includes a valve to allow the air pressure on the interior of the device to adjust to equilibrium with the ambient air pressure (not shown, see FIGS. 2 and 6B). In some embodiments, a valve can be included in the bottom portion of the device that is rigid. The valve can be depressed to allow the interior air pressure to reach equilibrium by way of a valve pin on the base 150 that presses against the valve or providing a user with a valve tool for depressing the valve. When the valve is pressed against the body of the device 100, near-airtightness can be achieved, thus allowing a pressure sensor, as described further below, to operate within acceptable error bounds. In some embodiment, a valve can be located in the top portion 105.

The valve may comprise a pressor surface for being pressed, which may be the same as or different from reset button 130.

LED 135 can be used to indicate status of the device 100 including, but not limited to, battery charge level, charging state, wireless communications status or activity, power state (on/off), error status, usage mode and the like.

Also included in device 100 is a battery or power storage unit 120 and charging receiving coil 125. Battery 120 can be a lithium-ion or some other type of rechargeable battery. Charging coil 125, likewise, can be a standard charging receiving coil. Charging coil 125 is used for induction charging. In some embodiments an added port for charging can make air pressure regulation more difficult. As a result, induction charging is preferred for charging battery 120.

Device 100 preferably fits in a standard adult-sized hand. Thus, the device 100 has a height 140 at its longest of preferably about 80 mm and a width 145 at its widest of preferably 60 mm. The device should have a diameter around 6 cm. The overall weight of the device 100 is preferably in the range of 90 g to 100 g.

Base 150 preferably includes the following components: wireless charging coil 155, electronics 160 for charging, a cooling system 165 to prevent overheating of the base during charging, LED 175 to provide charging status indication, and a power connector 170 for providing power for charging and LED operations. Base 150 preferably includes a hollowed volume matching the shape of the bottom portion 110 of the device 100 so that it remains stationary during charging.

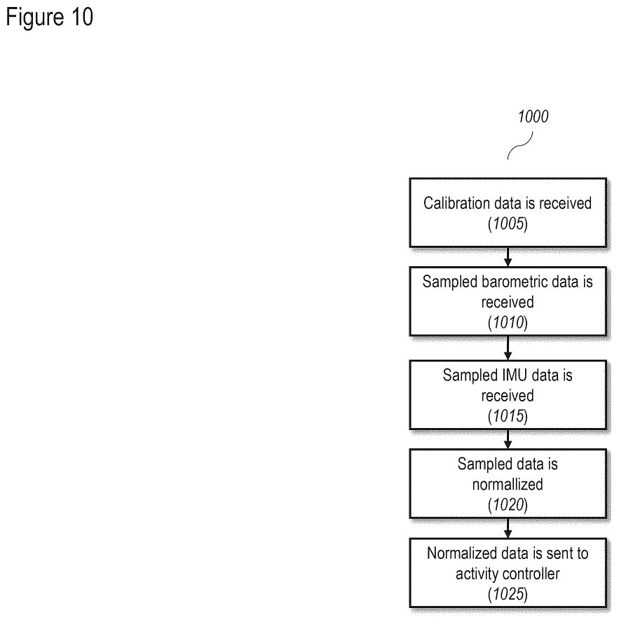

FIG. 2 illustrates an exemplary block diagram of a device 100 in accordance with embodiments. Device 100 includes a microcontroller 205 powered by battery 120 which is in turn charged with charging coil 125. Coupled to microcontroller 205 are various other components shown in the block diagram, preferably via PCB 115. Embodiments include one or more inertial measurement units (IMU)/sensor devices 220 for measuring orientation, position and movement of device 100. Preferred embodiments can use a sensor device such as the Physilog® from Gait Up, although other IMU/sensor devices can be used. IMUs that can be used include an accelerometer, a gyroscope, a magnetometer, and the like. A plurality of IMUs or different combinations of sensors can be used to provide tracking data with measurements of orientation, position and movement in multiple degrees or for more complex, compound movements for example, as described in U.S. patent application Ser. No. 16/172,818 which is incorporated by reference herein in its entirety or as described, for example, in connection with FIG. 10 including combining acceleration and magnetic field data from an accelerometer and magnetometer. Device 100 also includes a pressure sensor 225 coupled to microcontroller 205, such as a barometer, in accordance with preferred embodiments. Pressure sensor 225 is preferably a MEMS barometer given the preferred sampling rates, their size, cost, low power consumption, and the precision required for dexterous hand function measurement, rather than absolute grip force measurement. Pressure sensor 225 preferably measures force with a force sensitivity of <0.1 N and reliability of <0.1 N. Typically, the pressure inside the hand device will range from 0.7 bar to 1.7 bar. It should be understood that pressure sensor 225 can be combined with an IMU/sensor device 220 in that all of the sensors share a common housing and common electronics. For example, the preferred sensor device, the Physilog includes a 3D accelerometer, a 3D gyroscope, a barometer as well as other components described herein such as a wireless transceiver, as part of its family of sensors.

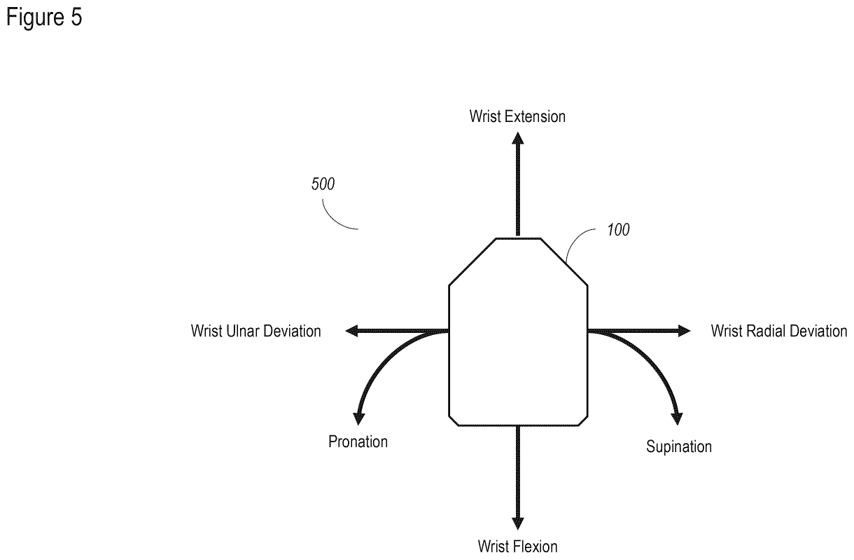

Referring to FIG. 5, an exemplary diagram 500 of movements of a device 100 measured by IMU/sensor device 220 is shown. A preferred embodiment will include at least measurement of wrist extension and flexion, wrist pronation and supination, and wrist ulnar and radial deviation. In particular, preferred embodiments will measure magnitude of displacement, velocity (which includes direction) and acceleration of the device in each of the directions indicated. Additionally, preferred embodiments will measure dexterous grasp of a user, including magnitude, velocity and acceleration of finger flexion (e.g., grip) and extension force in a pincer gesture, palming gesture, or whole-hand gesture. Gesture here refers to how the device is held in the hand. In some embodiments, the type of gesture is indicated by the activity performed by the user and, accordingly, the activity instructs the user on the gesture required.

Returning to FIG. 2, preferred embodiments can include a memory 230 for storage of computer instructions, buffering sampled data from sensor devices 220, 225, and the like. Wireless transceiver 235 can be used to transfer sampled data from sensor devices 220, 225 for analysis to an external computing device, to receive updates to software or firmware on the device, to receive instructions for executing computer instructions for adjusting power consumption, adjusting sensor sampling, operating a haptic motor, resetting or calibrating sensors, air pressure, and the like. To improve wireless connectivity, preferred embodiments will include an antenna on the upper face of the PCB 115. Some preferred embodiments can include a USB connector 240 to perform similar functions or for charging battery 120. For the same reasons that an induction charging coil is preferably used to charge a device rather than a different kind of charging port, wireless communication is preferable to a USB port.

FIG. 2 shows a reset button 130 and valve 250 as separate components. It should be understood that the reset button 130 and valve 250 can be combined so that the reset button 130 also adjusts the interior pressure to the ambient pressure when pressed.

Some preferred embodiments can further include a haptics motor (not shown) for providing haptic feedback to the user during an assessment, therapy, or other activity.

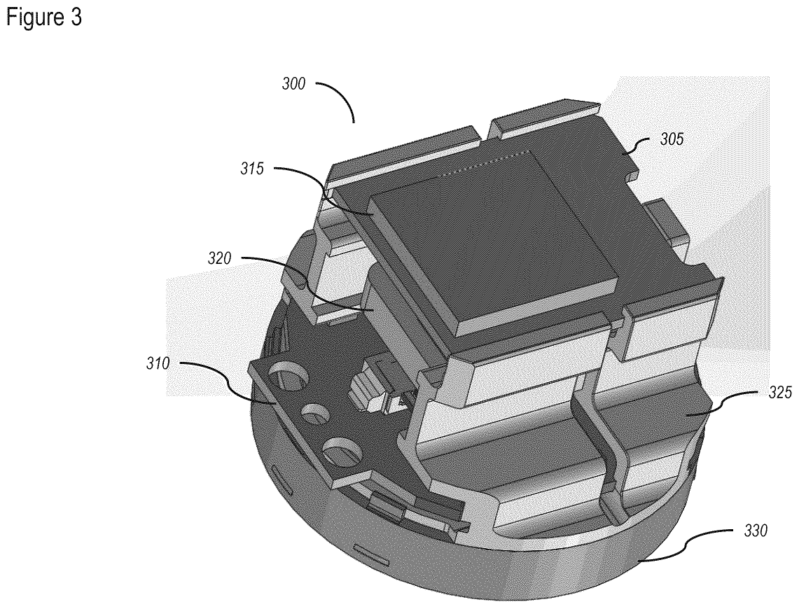

FIG. 3 illustrates an exemplary design 300 for the electronics of device 100 according to some embodiments, the design 300 can include two PCBs 305, 310. A sensor device 320 is placed on PCB 305 while microcontroller 315 is placed on PCB 310. Housing 330 contains a battery and charging coil. Structural members 325 hold the various components in place and maintain stability in relation to the device body.

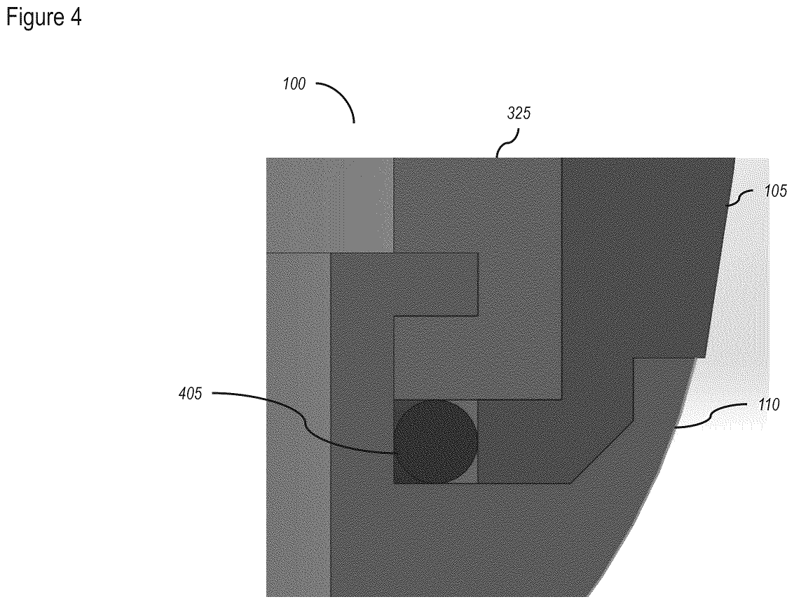

FIG. 4 illustrates an exemplary design cross-section of a device 100 in accordance with embodiments. In the figure shown the top portion 105, bottom portion 110, and housing 325 are fitted together in a jigsaw fashion with an O-ring 405 to maintain a seal at the confluence of the three components. The O-ring 405 can have different cross-section shape profiles so long as a seal is maintained. It should be understood that, as noted above, the seal is intended to be near-airtight, but non-hermetic to provide for ease and cost-effectiveness of manufacturing, repair and the like.

Referring now to FIGS. 6A and 6B, exemplary illustrations of a device 600 in accordance with embodiments are shown. Device 600 includes an active marker 607 for use in tracking applications. By including active marker tracking in the device along with sensor tracking accuracy of movement and position is increased. An active marker 607 can be an active marker as disclosed in U.S. patent application Ser. No. 16/524,085 which is incorporated by reference herein in its entirety. Embodiments that include an active marker can include a third portion 605 in addition to a top portion 610 and bottom portion 615 similar to the top and bottom portions as discussed in connection with FIG. 1, the third portion 605 being above the top portion 610 and housing the active marker 607. In some embodiments, the top portion 605 can include a semi-transparent or semi-opaque material such that active marker 607 can be made visible from underneath the material of the top portion 605. The active marker can be powered via the same power storage device providing power to the other components of device 600. The exemplary device 600 of FIG. 6 has a height of 79.96 mm. Device 600 also includes a reset button 620 and two LEDs 625. LEDs 625 can be used in combination to communicate status indicators described herein.

In some preferred embodiments, the device surface may include high contrast graphical elements for possible computer vision tracking, calibration, or both, in lieu of or in combination with an active marker. Green or blue are preferrable colors for segmentation from hand detection. To the extent the device 100 or 605 includes such graphical elements, such graphical elements are preferably included on the top portion 105 or 610, respectively.

FIG. 6B illustrates the bottom face of the bottom portion 615, showing placement of the reset button 620 and LEDs 625. Also shown is a valve 635 for regulating air pressure as described herein. In preferred embodiment, the bottom portion 615 includes a sealed cover for an opening 640 to the underside so that some electronic components are reachable. In some cases, a hand device can accept a standard alkaline battery or a type of rechargeable battery, but without a charging coil and thus requires access to the battery compartment.

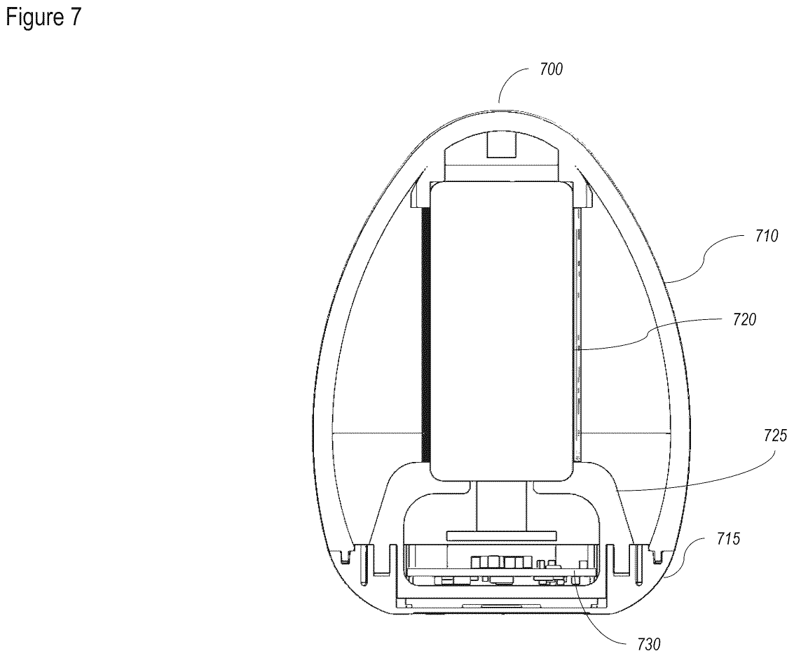

Turning now to FIG. 7, an exemplary design for a device 700 in accordance with embodiments is illustrated. Here, a sensor device 720 is oriented orthogonally to PCB 730 and is held in place via structural members 725. PCB 730 is placed horizontally along the base of the device 700.

Embodiments of hand devices described herein can allow detailed assessment of a user's dexterity via simple, lightly gamified activities designed to output accurate metrics of performance. Such activities can be provided by the therapy platform described above.

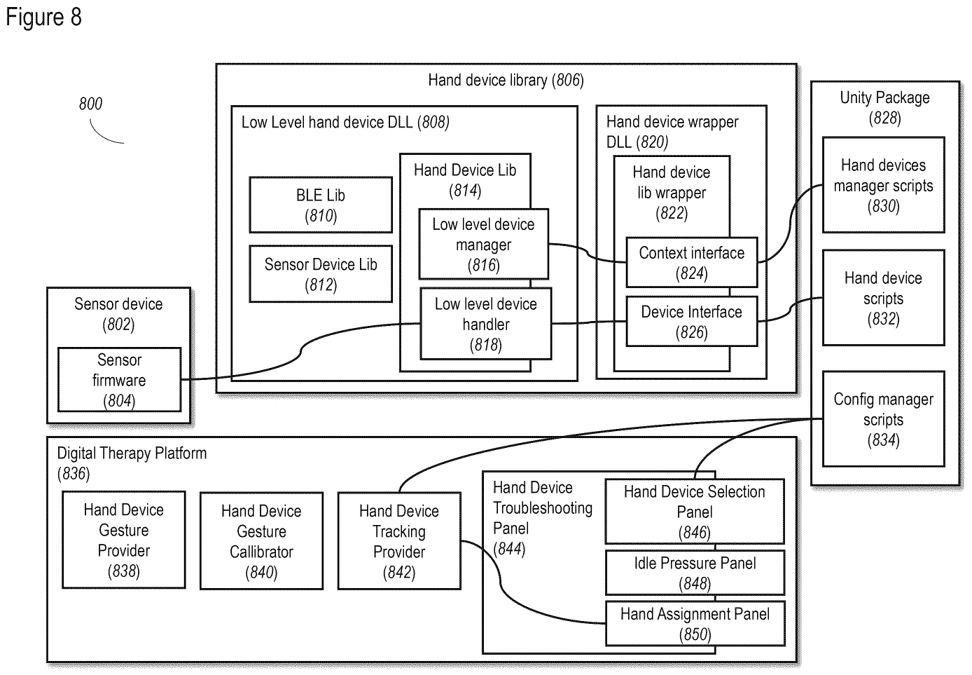

Hand function assessment, training and therapy, including finger individuation and dexterous grasping, for hemiparesis and other conditions using embodiments of the device described above is useful in the context of a larger digital therapy platform. Such a digital therapy platform can include serious games for training and therapy and assessment activities. One such a digital therapy platform is the MindMotion™ platform from MindMaze SA. FIG. 8 illustrates a block diagram of an exemplary architecture 800 for integrating hand function devices as described herein with such a digital therapy platform. It should be understood that the architecture presented is applicable to a PC computing environment, to a distributed computing environment or to any other suitable computing environment. Those of ordinary skill in the art understand how a similar architecture in other computing environments, operating systems or platforms would be organized.

A hand device library 806 preferably includes two run-time libraries. The low level hand device DLL 808 includes a BLE lib 810, for hardware/OS abstraction for low level access to wireless communication services, e.g., Bluetooth: a sensor device lib 812 for generic sensor device communication protocol and configuration methods: and a hand device lib 814 for configuration and setup specific to the hand device. In the embodiment illustrated, the low-level device handler 818 is in communication with the sensor firmware 804 to handle connection and streaming with the sensor device 802. In preferred embodiments, streaming data parameters can be set on connection. Streaming data parameters can include selection and configuration of packet content, including timestamp data, acceleration data, gyroscope data, barometer data, temperature data, data normalized according to calibration, quaternion data, and various calibration parameter data for IMUs, including accelerometers, gyroscopes, and the like. Calibration parameter data can include gain, offset and scale. It should be understood that packet contents should be configured according to the sensor devices used in the hand device connected. The other run-time library, the hand device wrapper DLL 820 includes a wrapper for the hand device lib 822 interface. Accordingly, the context interface 824 provides an interface for the low-level device manager 816 and the device interface 826 provides an interface for the low level device handler 818.

Unity Package 828 includes software components including hand devices manager scripts 830, hand device scripts 832, and config manager scripts 834. The hand devices manager scripts 830 creates game and activity objects when a hand device is discovered and handles messaging communication generally with the hand devices through the context interface 824. Hand devices scripts 832 provides configuration and data management for an individual, identified hand device through the device interface 826. The config manager scripts 834 provides startup and live configuration of a set of hand devices by automatically discovering, connecting and streaming when necessary. Unity is a preferable scripting tool appropriate for the exemplary architecture 800 and it should be understood that other development platforms can be used.

The exemplary digital therapy platform 836 shown includes unity scripts in a hand device gesture provider 838, hand device gesture calibrator 840, hand device tracking provider 840, and a hand device troubleshooting panel 844. The providers 838, 840 and 842 provide services for their respective activity functions (i.e., calibration, tracking, etc.). For example, tracking provider 840 provides device tracking capabilities for activities and games (not shown) in the digital therapy platform 836. The troubleshooting panel 844 provides services for fixing connections, hand assignment for devices, and fixing reset issues for hand devices. In the embodiment represented, the troubleshooting panel 844, includes a hand device selection panel 846, idle pressure panel 848, and a hand assignment panel 850 to provide those and other services. For example, the selection panel 846 can communicate with the config manager scripts module 834 to ensure a sufficient number of devices are streaming for the current activity in the platform 836. The idle pressure panel 848 captures the air pressure of a hand device while idle. Preferably a command is provided to the computing device operating idle pressure panel 848, to indicate that the hand device is idle (not being manipulated by a subject). The hand assignment panel 850 can assign a left or right hand to a device for the hand device tracking provider 842.

The architecture described in connection with FIG. 8 is exemplary and applies to some preferred embodiments. Additionally, the components and their organization are illustrative, and they can be further separated or combined in accordance with embodiments. In some embodiments, some components can be implemented using different technology. For example, Windows-based components and Unity, which is a commonly used development platform, are described in the exemplary architecture of FIG. 8, but other platforms can be used and those of skill in the art can understand how target systems other than Windows can support embodiments.

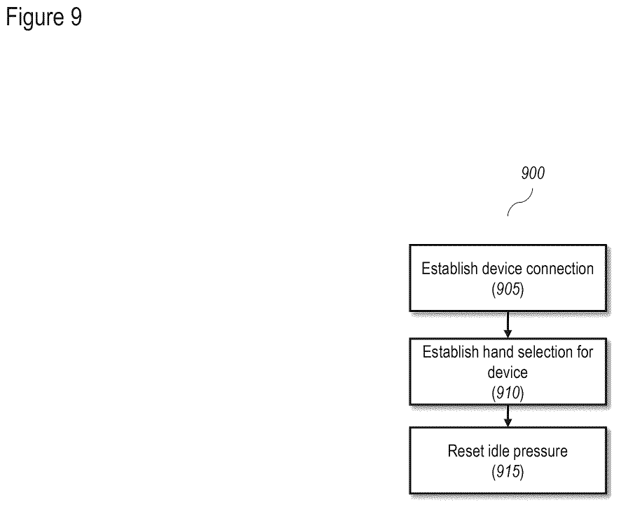

FIG. 9 illustrates a flowchart 900 of a method for initiating a hand device for use with a therapy platform in accordance with embodiments. In step 905, a connection to a hand device is established. In some embodiments a connection to a hand device can be made according to a known wireless protocol, e.g., Bluetooth, for establishing a connection with a peripheral. In step 910, a hand selection indicator is selected for the device. The device is assigned to either a left or right hand so that tracking is accurate for the therapy platform activity. In some instances, an activity will call for the use of two hand devices, each of which can need to be assigned to a specific hand. In yet other cases, an activity can call for more than one user, each using one or two hand devices. Accordingly, a plurality of hand devices can be assigned to the same hand side (i.e., left or right). Such assignment is important so that particular movements can be measured properly such as wrist pronation and supination, wrist extension and flexion, and the like. In some embodiments, this step can be performed by simply receiving an instruction having a hand-side indicator which can be generated from manual user input or from detection of specific movements. For example, an instruction to request a wrist extension, or some other movement, can be sent to prompt the user to move the hand device using that movement. The sensor data indicating the direction of movement or displacement of the device can reveal which hand is being used: and the hand-side indicator can, thus be determined.

At step 915, the idle pressure of the device is reset. In preferred embodiments, the air pressure of the hand device is captured from a barometer reading when the hand device is not in use or in the grasp of a user. As noted above, preferred embodiments of the hand device are non-hermetic and, thus, the air pressure is likely to differ from one use to the next. Thus, the internal air pressure is preferably read to establish a baseline prior to commencement of an assessment or therapy exercise.

FIG. 10 illustrates a flowchart 1000 of a method for providing hand device sensor data for use in an activity in a therapy or other platform. At step 1005, calibration data is received. In preferred embodiments, data for user range of motion, maximum pressure for gestures, minimum and maximum acceleration, and the like are received from a calibration process during which the user performs certain movements, grips and the like. The calibration data is retained in a data store, for example, a memory such as in FIG. 2, for later retrieval during an activity. Range of motion calibration is typically made in world space, not in local space. As an example, range of motion data can be determined from accelerometer data. For wrist flexion/extension, a plane is derived from an acceleration vector when the device is at maximum flexion and one when it is at maximum extension. The angle between these two vectors on the plane then can give the range of motion in degrees. At step 1010, raw sample barometric data is received. At step 1015, raw IMU sensor data is received. The raw data for each of these steps preferably includes timestamp data. It should be understood that the sensor data received can be from one or more different sensors of the same type, different type, or a combination thereof. For accelerometer data, preferred embodiments include a signal processing to dampen the signal with minimal impact on latency for use during calibration and activity use.

Embodiments of the present invention can be used to measure and track complex, compound movements based on accelerometer readings. For example, a movement involving yaw measurement from acceleration data is not possible without having a corresponding measurement of gravity in a device in typical devices. Such a movement involves, for example, wrist extension while holding the device as it rests along a flat surface (e.g., a table). Using an advanced gyroscope can provide readings to create controller data for an activity. The inventors have found that combining measurements from an accelerometer with magnetometer readings can be used as well to maintain an overall lower cost device.

The state of the art to compute the orientation of a device from an IMU is by using mainly the gyroscope signal. In practice, however, gyroscope signals have an inherent drift, the amount of which is highly dependent on the sensor. This drift can lead to an incorrect orientation reading after a few minutes and, therefore, can require frequent recalibration by the user. Even the best gyroscope-derived quaternion in the state of the art has drift of a few degrees per minute Accordingly, in some preferred embodiments an accelerometer reading can be more appropriate for some activities, particularly serious games where the hand device is used as an activity or game controller. Accelerometers generally give a drift-free signal, but an accelerometer gives information about just two axes, pitch and roll. The pitch and roll are computed assuming that the device is static, any movement with an acceleration will have an impact on the computed orientation. Preferred embodiments, therefore, project the current acceleration vector on this plane and compute the angle between the projection and one of these two vectors to give the current position. As a result, most of the “jerkiness” is mitigated. The filter can be tuned according to needs for certain activities with a trade off with latency.

At step 1020, the raw data is normalized. This step preferably uses data about which hand, which user, the type of gesture (e.g., the type of grip used), calibration data, and other configuration data to normalize the data into values useful for an activity. In some instances, normalization can include increasing magnitude of values of some of the data in relation to other data based on a user's range of motion. The user's range of motion parameters can be received from, for example, step 1015 during which the user is prompted to perform certain movements, grips, etc. For example, the magnitude of finger flexion can be increased in relation to wrist flexion where the user's finger range of motion is reduced but wrist range of motion is not so that a finger flexion appears more sensitive in the context of an activity. Preferred embodiments can also normalize pressure data against temperature readings from the interior, exterior or both of the device in cases where the pressure sensor does not already compensate.

At step 1025, normalized data is sent to an activity controller that is part of a therapy or other activity platform. For example, in preferred embodiments the normalized data can be sent to a hand device gesture provider 838 (for pressure data), hand device tracking provider 842 (for IMU data). Other types of activity controllers can receive normalized data depending on the type of activity platform.

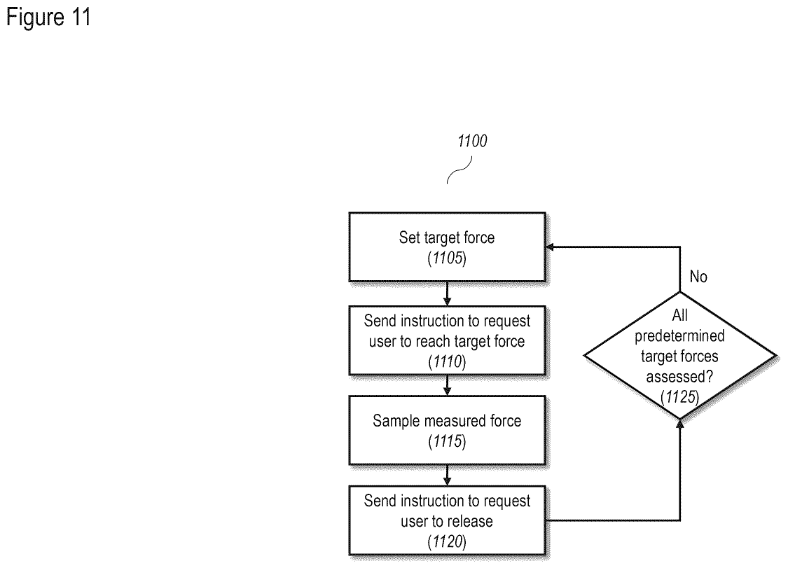

FIG. 11 illustrates a flowchart 1100 of a method of assessment for grasp and release measuring force. At step 1105, a target force is determined against which the user's grasp is tested. At step 1110, an instruction is sent to provide a prompt to the user to begin applying force to the device. In preferred embodiments, the user is requested to apply the force for a determined amount of time. When instructed, the user squeezes the device to reach a measured force, maintain that force for a short period at the target force, then completely release it. In such cases, the force applied by the user is measured by way of detecting changes in the barometric pressure of the interior of the device, for example at step 1115, so that it can be determined whether the user was able to reach the force and how well the user was able to maintain the force over the amount of time. The metric from this assessment is a function of the delta between the force applied and the target force. This metric provides information on the range of forces within which the patient is able to accurately grasp and maintain his grip. At step 1120, an instruction is sent to provide a prompt to the user to release the grip. At step 1125, the process is repeated for a range of target forces. In some cases, the range of target forces can include only one target force. In other cases, the activity assesses this through a large range of forces, from gripping the device very lightly to squeezing it tightly.

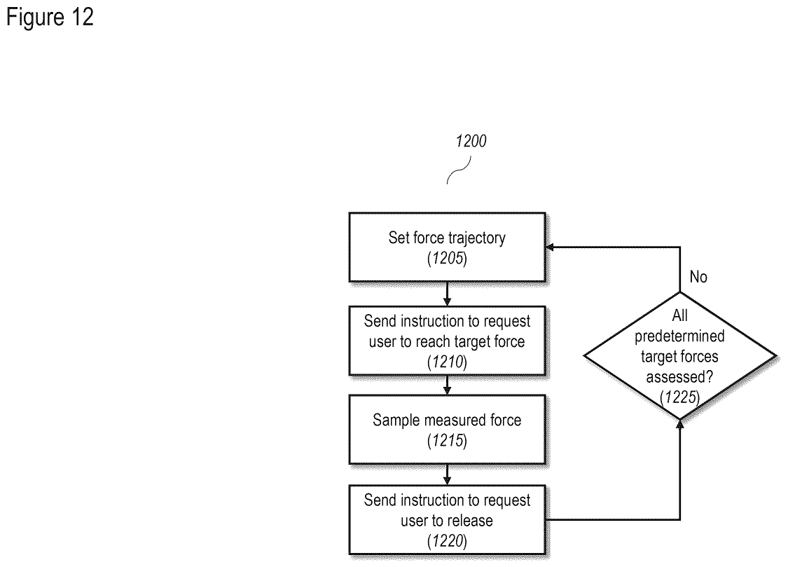

FIG. 12 illustrates a flowchart 1200 of a method of assessment for grasp and release measuring modulation. At step 1205, a target force trajectory is set. The assessment calls for the user to regulate the force applied to the device in order to follow a trajectory. The assessment trajectory can include target forces that form a sinusoidal or some other waveform when plotted on a y-axis against a time x-axis or that are irregular or random. At step 1210, an instruction is sent to provide a prompt to the user to begin applying force to the device. At step 1215, the barometric pressure is sampled as the target force changes along the trajectory. At step 1220, an instruction is sent to provide a prompt to the user to release the grip and end the assessment. The assessment metric is a function of the delta between the trajectory that the player traces by squeezing/releasing the device and the target trajectory. Target trajectories will have regions where the patient will have to modulate grasp with varying frequency and amplitude. This provides a measure of how accurately the patient can modulate his grasp force as a function of force applied and grasp/release frequency.

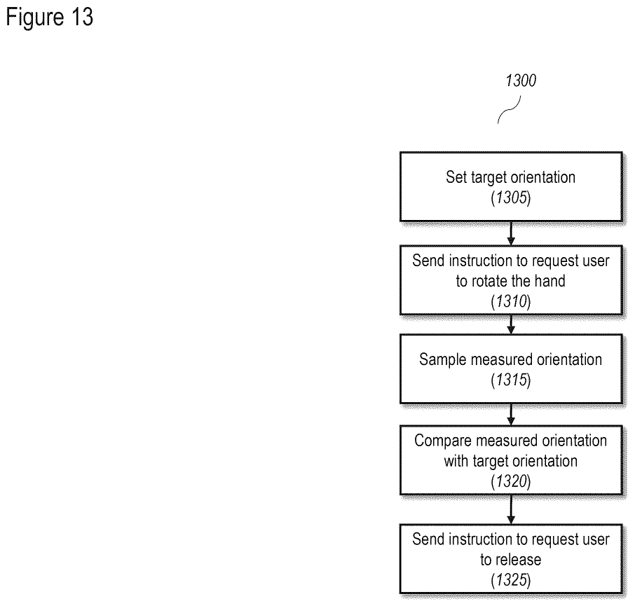

FIG. 13 illustrates a flowchart 1300 of a method of assessment for pronation and supination. At step 1305, one or more target orientations are determined. At step 1310, an instruction is sent to prompt the user to rotate a hand while holding the device. According to preferred embodiments, the user would rotate the device as instructed, using pronation and supination of the hand. At step 1315, the orientation of the device is sampled from the IMU. At step 1320, the orientation is compared with the target orientation. The angular delta between both orientations throughout the assessment will be used as a measure of the patient's ability to accurately pronate/supinate the wrist throughout a range of rotational amplitudes and speeds. At step 1325, an instruction is sent to prompt the user to end the assessment. Again, the steps from step 1310 can be repeated for additional orientations. In preferred embodiments, the sample orientation data is used generating user interface elements, for example, on a monitor or in an AR/VR environment to assist the user in reaching the target orientation.

In each of the methods described above and generally during use of the device, a sampling rate of at least substantially 60 Hz is used for both IMU and pressure sensor. More preferred is a sampling rate of substantially 100 Hz for both IMU and pressure sensor. Most preferred is a rate of 120 Hz. Sampling rates can differ for the IMU and pressure sensor, however. In some preferred embodiments, the sampling rate can be decreased for a sensor during use cases where the sensor readings are less critical. For example, in cases where the device is used to measure primarily force metrics, but not orientation, the IMU sampling rate can be throttled. Additionally, sampling rate can be throttled to conserve power, either automatically or based on a user setting.

The methods described above in FIGS. 11-13 can be combined in some preferred embodiments, for instance having to modulate one's grasp while also performing pronation/supination. That is, some exemplary embodiments include a method tracking hand and wrist movements with dexterous hand function metrics simultaneously using markerless tracking or IMU sensor data and pressure data.

The assessment methods describe above can allow each patient to be represented as a point in a multi-dimensional “impairment space,” with coordinates corresponding to the score associated with each metric. For example, one of the axes might correspond to “low-force grasp-and-release accuracy score.” Collecting representative data on healthy subjects will allow the identification of a “healthy cluster” within this space (corresponding to the typical scores across all assessments of healthy subjects). The patient's impairment can then be quantified as a function of the distance between his location in impairment space and the centroid of the healthy cluster.

Furthermore, the assessment methods can be used longitudinally in order to track a user's recovery or progress through a change of position in impairment space, quantifying the efficacy of a user's therapy plan as a function of how quickly the user's location approaches the healthy cluster.

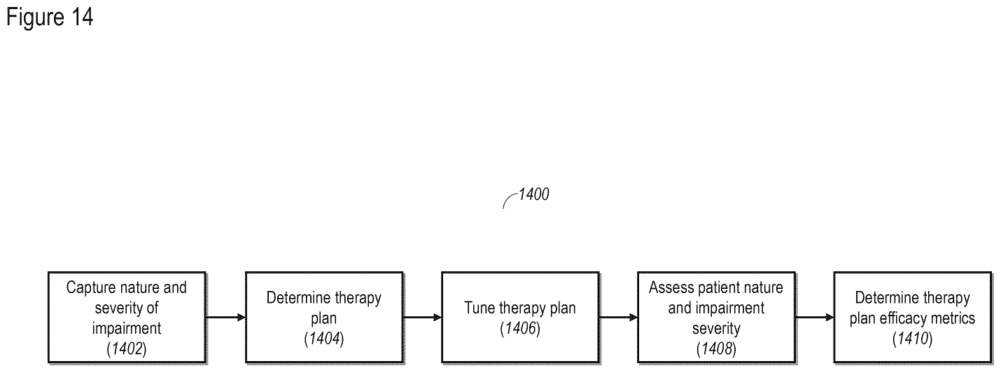

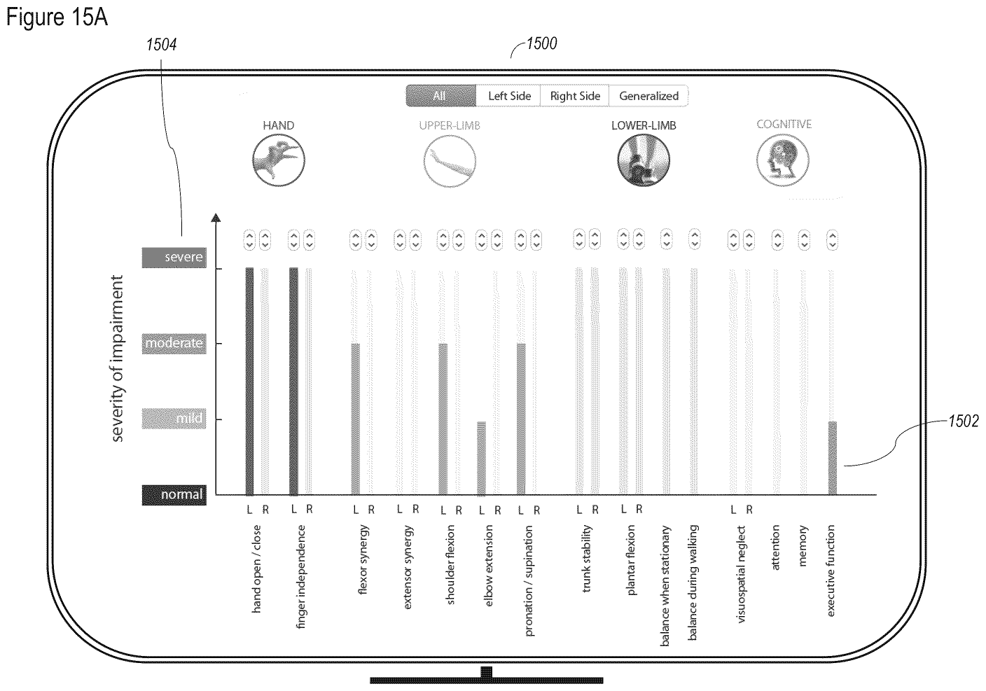

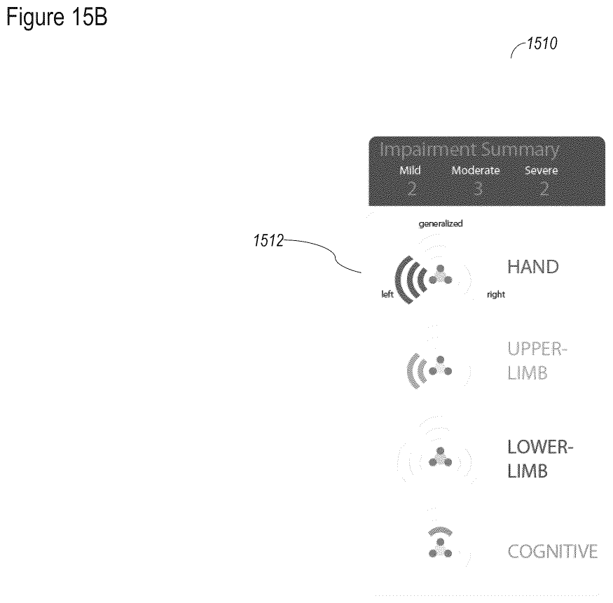

The hand device disclosed herein as well as other devices whether or not known or state of the art device are particularly useful in an assessment and therapy context as described below. For example, FIG. 14 illustrates an exemplary flowchart 1400 for a digital therapy protocol in accordance with embodiments. At step 1402, the nature and severity of a patient's impairment are captured. A therapist registers a new patient in MindMotion. The system can request that the therapist rank a patients' severity along a set of predefined measures of clinical impairment. On a macro-level, these impairments can correspond to behavioral functions (e.g., hand, upper-limb, lower-limb, cognitive, and the like). These functions, in turn, can be composed of fine-grained measures of impairment. In some embodiments, the system can accept measures input by the therapist. In some embodiments, the measures can be determined through an automated assessment process that corresponds to step 1408. The system can incorporate a user interface for the input and display of such measures, as in FIG. 15A, which illustrates an exemplary input and display user interface on display device 1500. The user interface includes values 1502 for fine-grained functions which can include, but is not limited to, such functions as hand open/close (left and right hand) and finger independence (left and right hand) for hand: flexor synergy (left and right upper limb), extensor synergy (left and right upper limb), shoulder flexion (left and right upper limb), elbow flexion (left and right upper limb) and pronation/supination (left and right upper limb) for upper limb: trunk stability (left and right lower limb), plantar flexion (left and right lower limb), balance when stationary and balance during walking for lower limb; and visuospatial neglect (left and right side), attention, memory and executive function for cognitive. The values 1502 can be based on a scale 1504. FIG. 15A shows a four-value scale of normal, mild, moderate and severe although other scales can be used which are well-known to those of ordinary skill in the art. FIG. 15B illustrates another exemplary user interface 1510 for input and display of impairment assessment measurements. Measurement values are illustrated and can be set graphically using. for example, intensity bars 1512 as shown that depict both the value and side affected. In the user interface 1510 the scale used includes values mild. moderate and severe. It should be understood that impairments and/or functions and scales other than those shown can be used in some preferred embodiments.

As the impairment features are automatically or manually populated, a macro-level summary of the patient's nature and severity of impairment is visualized for display as a user interface for a therapist or other user (e.g., a patient). In preferred embodiments, the system presents other options for the therapist to define or further customize a therapy plan. For example, the system can receive start and end dates of the patient's visits or sessions, the average time per therapy session and the like. Embodiments can then use this information as input to customize a therapy protocol or generate recommendation data for a therapy protocol.

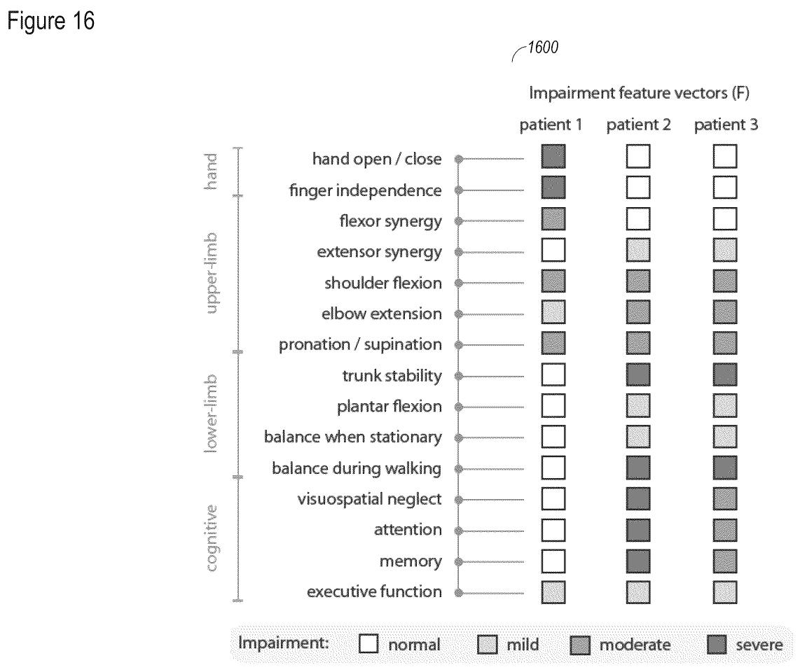

Referring now to FIG. 16, an exemplary illustration of impairment function vectors for different patients is shown. Each patient has a function vector profile comprising multiple functions with a scale value for each dimension in the vector. In some embodiments, a vector can include null values for functions that are not assessed. In other words, some preferred embodiments can accommodate assessment data or measurements from a subset of the functions that the system can accommodate. Behind the scenes, a patient's impairment and severity can now be represented as a mathematical impairment feature vector (F). Impairment feature vectors for two patients might look very distinct (patient 1 vs. 2) or very similar (patients 2 vs. 3). Some preferred embodiments will generate different treatment plans for distinct feature vectors, (patients 1 vs. 2) and similar treatment plans for patients with similar feature vectors (patients 2 vs. 3).

Returning to FIG. 14, at step 1404, a therapy plan is determined. The idea is to provide the best therapy plan prescription for a patient based on his/her nature and severity of impairments. As described above in relation to step 1402 or below in relation to step 1408, a therapist enters metrics or a patient performs a multi-modal assessment which results in a vector of the patient's impairment F(f1, f2, . . . , fn) for a particular function. Each component fi contains the impairment level associated: normal, mild, moderate or severe. In preferred embodiments, a holistic multi-modal assessment is performed to generate a richer set of metrics than is available in the current state of the art. Current assessment and therapy techniques treat functions such as those discussed herein as unrelated in that functions are assessed and treated independently. For example, a peg board test is a common assessment tool intended for hand function impairment. The nine-hole peg test can be affected by upper limb and cognitive impairments. Moreover, the nine-hole peg test fails when used repeatedly for a patient because the patient adapts cognitively and can use compensatory movements to improve rather than isolating hand function. Even if such a test is biased in favor of the intended function, the extent of bias is unclear, particularly across patients and even from session to session for the same patient. The same phenomenon exists in therapy. That is, a therapy is nominally aimed at a function but affects other functions, perhaps to the detriment of the nominally aimed function. Similarly, a therapy can be nominally aimed at multiple functions but is biased towards only a subset of the functions. The impairment vector groups functions quantifies the patient's impairments so that proper therapies can be determined taking into account the biases of both the assessments and therapies used by the therapist.

The goal is then to suggest an optimal therapy plan matching the patient's impairment vector by using a combination of pre-defined “digital therapies.” In preferred embodiments, digital therapies can include activities performed by patients that correspond to known exercises. In some cases, a digital therapy can include an exercise that isolates a function to optimally remove bias towards other functions. In some cases, a digital therapy can include an exercise that targets specific co-occurring impairment types (e.g., mild hand open/close+pronation/supination deficit). Each digital therapy can be either a single gamified activity or a set of activities taken from the activity catalogue. In preferred embodiments, a digital therapy is associated with a vector P(p1, p2, . . . pn) where each component pi can be a scale value as described herein.

A linear regression is performed in order to find what is the best combination of digital therapies for each patient using vectors Pi based on his/her impairment vector F. Various linear regression models could be used to determine the relationship between the impairment vector F and the optical therapy vectors Pi such as a least squares regression analysis. Once the optimal combination of digital therapies is identified, a decision is made on the number of digital therapies to combine in the treatment plan as well as rating the order of importance (e.g., severe hand impairment is the primary focus and then moderate subluxed shoulder). This decision is based on the highest weights β derived from the linear regression, the severity of the impairment addressed by the therapy plan and medical information of the patients (e.g., condition, time of onset, pain and the like). An optimal therapy therefore can be derived by the following:

$$patient impairment vectors ( F ) = β \times P \qquad ( 1 )$$

$$optimal therapies ( β ) = {( {P}^{T} P )}^{- 1} {P}^{T} F \qquad ( 2 )$$

Some preferred embodiments allow a therapist or other user to input schedule and dosage (i.e., duration of session and the like) data. In some cases, schedule and dosage data can be determined from modeled data from, for example, step 1410. In other cases, schedule and dosage data can be set to standard or generally accepted values from guidelines or the current state of the art. Other factors can include the patient's condition or insult (e.g., stroke, TBI, etc.), age of the conditions (e.g., number of years/months, chronic/acute, etc.), a severity level, number of relapse episodes (e.g., for multiple sclerosis), and the like. Those of skill in the art can appreciate various factors that could be used to adjust schedule, dosage or both.

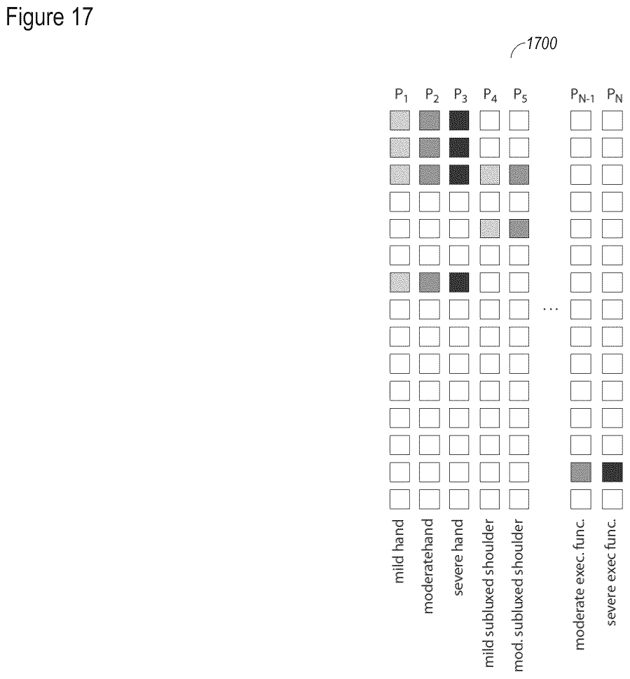

FIG. 17 illustrates an exemplary set of predefined digital therapies with scale values. As shown, each therapy substantially mirrors the structure of an impairment vector in that each function within a therapy is assigned a scale value. In preferred embodiments, the scales for the impairment vector and the therapy vector are identical although some embodiments can accommodate different scales wherein a correlation between the scales is defined. These protocols are defined either by scientific literature or have been identified by experts in the field (therapists/neurologists), and usually target specific co-occurring impairment types (e.g. mild hand open/close+pronation/supination deficit). The system then estimates the combination of protocols that best explain the patient's impairment prole. The identified combination of digital therapies, coupled with a suggested schedule and dosage is then the recommended therapy plan for the patient. In the example shown, digital therapies P₂ and P₄ could correspond to patient 1's impairment vector and, accordingly, by prescribed to patient 1.

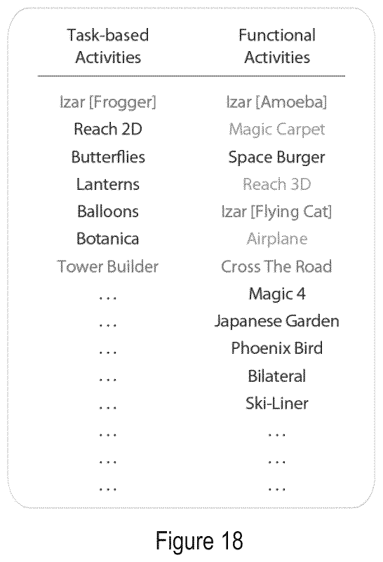

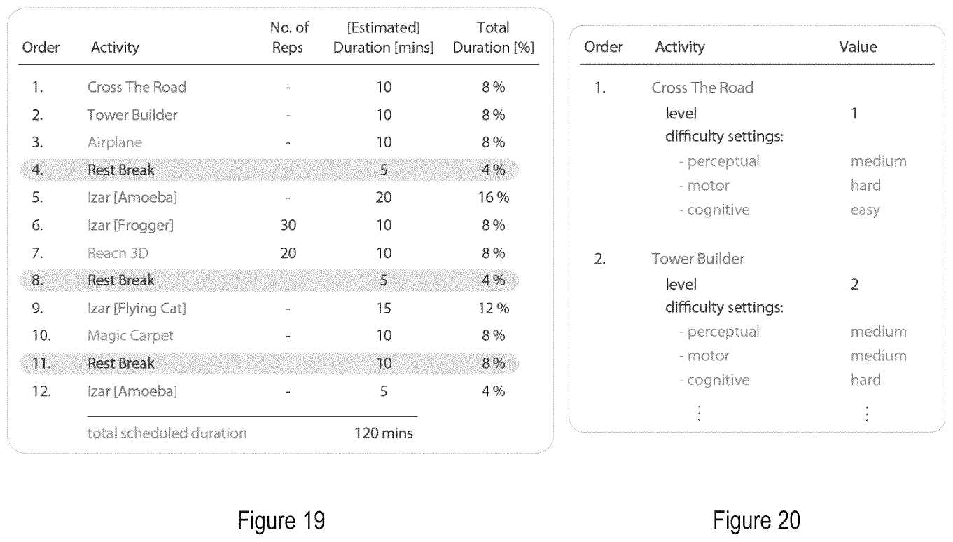

In preferred embodiments, the optimal digital therapies are then used to determine the activities or exercises to prescribe to the patient. Activities that are appropriate for the patient for the digital therapies are identified and selected. In some cases, activities, which can be task-based and functional, are developed with a single therapeutic goal in mind. Thus, it is straightforward to identify a set of activities from within a set of predefined activities that are appropriate to train the specific impairment features (e.g., activities that train severe hand and moderate subluxed shoulder impairments for patient 1 from FIG. 16). In some preferred embodiments, each of the digital therapy plans within the system can have a predefined therapy plan structure of activities versus rest phases, that the chosen activities can then slot within. FIGS. 18, 19 and 20 illustrate an exemplary catalog of activities, a therapy plan structure and detailed view of a therapy plan structure, respectively.

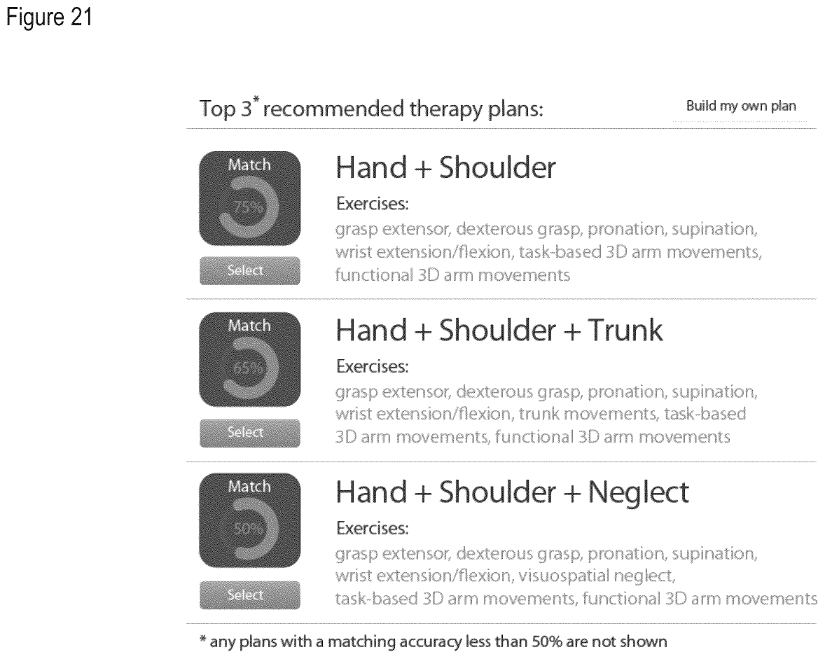

FIG. 21 illustrates an exemplary user interface view of recommended digital therapy plans. Preferred embodiments determine a match level for each therapy with respect to the patient's impairment vector. The match level indicates the correspondence derived from the algorithm for determining the optimal therapy plan.

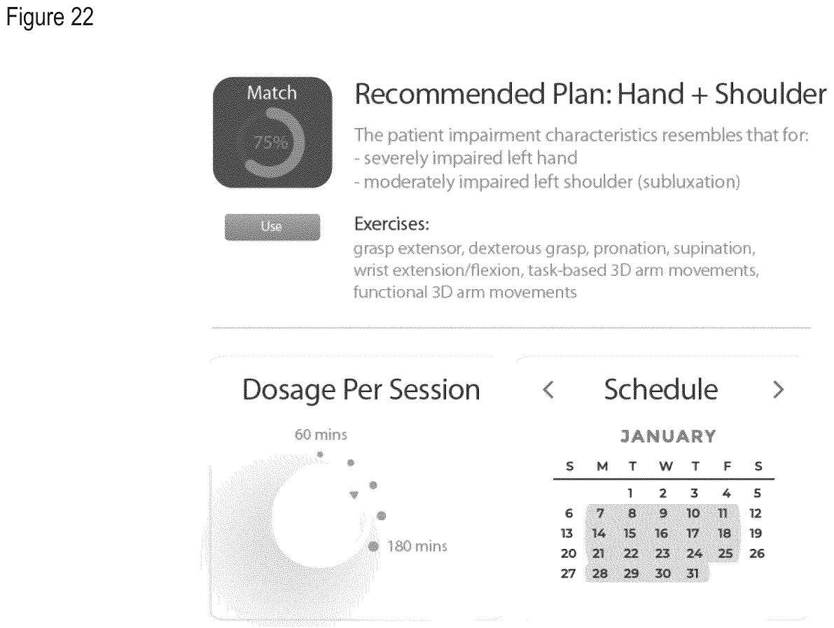

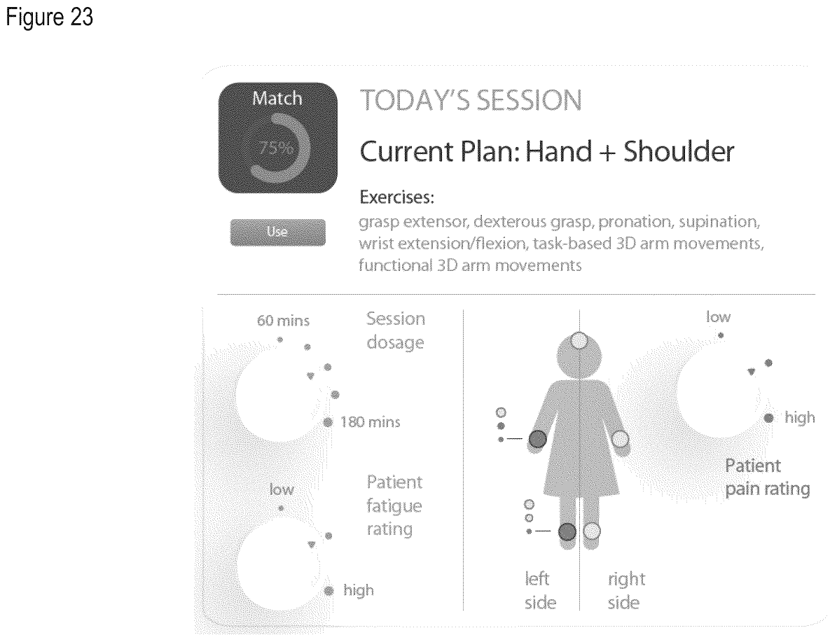

After a therapist enters (or automatically assesses a patient's impairment prole), all the work done to estimate a therapy plan (appropriate activities+schedule+dosage) is done behind-the-scenes. At this point, the therapist is shown the top three digital therapy recommendations that best explain the patient's impairment prole. The therapist is fully in control of selecting whether they want to go with one of the recommended plans, or customize any of the plans, or completely build a therapy plan from scratch by themselves. The interface is designed to provide only the key parameters that the therapist requires to understand and chose a plan (appropriate activities/exercises, schedule & dosage). Prior to starting each session associated with a plan, the therapist is able to modulate the session time (within reason) and enter the patient's current fatigue and body-part pain ratings. FIGS. 22 and 23 illustrate exemplary user interfaces for adjusting such parameters.

At step 1406, the therapy plan is tuned. Despite best efforts, the therapy plan might not be fully matched to a patient's impairment level. If individual activities within the therapy plan are too easy or too difficult, then the patient will become demotivated, leading to reduced time-on-task. Thus, at this step, embodiments can provide parameters to a therapist or other user to modulate the difficulty of individual activities within a therapy plan to the nature and severity of the patient's impairment profile.

The production of any complex sensorimotor task relies on the action-perception loop. Patients will perceive information, process it to produce motor output in order to interact with the world. In the case of digital-based activities as those provided by embodiments as described herein, the difficulty of the activity or game is further influenced by the nature of the patient's impairment (e.g., motor vs. cognitive). Thus, preferred embodiments include parameters controls based on the nature and severity of the patient's impairment.

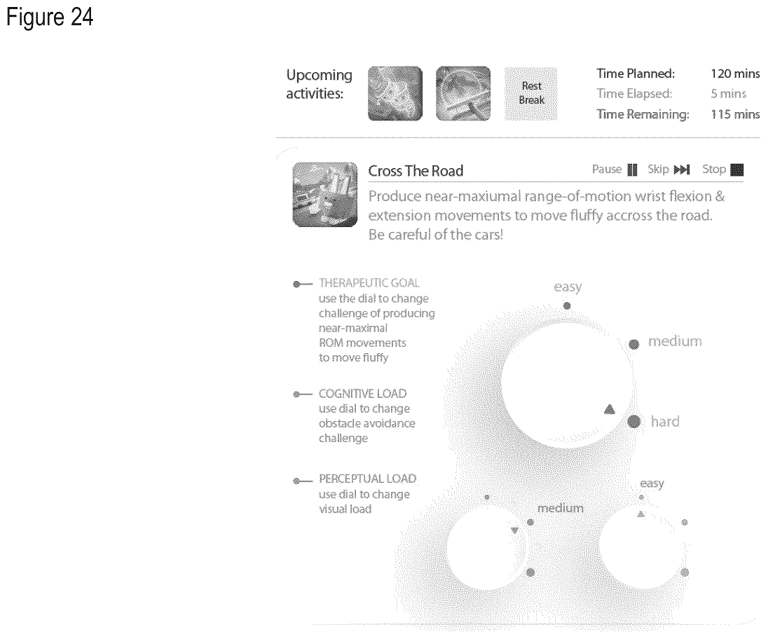

To enable this modulation, preferred embodiments display one or more parameter options in a user interface for a user to set a value. The values are then used to adjust aspects of the activity. Each activity has a specific set of one or more parameters that influence the difficulty. Despite all games being influenced by the interaction between the action-perception loop and the patient's impairment profile, since the game logic of each activity is different, the parameters that influence each game's difficulty are different. In the most extreme case, the parameters for two games might be completely different. Thus, what is required is the flexibility of designing activities with different game logics, while providing a unified experience for therapists in order to control the difficulty of a wide portfolio of disparate activities. In preferred embodiments, the user interface includes 3 elements that are consistent across all activities and that correspond to the elements of the action-perception loop (perceptual load, cognitive load, motor output). Each activity parameter maps in an individualized way onto the user interface elements. In some embodiments, the user interface elements for the difficulty parameters can have limited number of settings for ease of use. An exemplary illustration of a user interface in accordance with the difficulty parameters in such an embodiment is shown in FIG. 24.

At step 1408, the patient's nature and impairment severity are assessed. Judging the efficacy of a therapy plan requires that a patient's impairment profile is measured at frequent intervals and compared to pre-intervention baseline. In some preferred embodiments, assessment metrics can be received by the system during activities for therapy of purpose-built games that capture the relevant impairment metrics during the appropriate movements required within the activity. The table below provides an example of the corresponding impairment, assessment activity, and source of the impairment metrics that can be received during activities.

| Impairment | Assessment Activity | Peripheral |
|---|---|---|
| Hand Open/Close | Gamified grasp | Hand device for |
| | flexion/extension | quantifying grasp force |
| | task | |
| Finger | Gamified finger | Finger individuation device |
| Independence | individuation task | |
| Flexor Synergy | Gamified 3D reach | Multi-muscle recording from |
| | to grasp task | upper-limb extensors + |
| | | flexors |
| Extensor Synergy | Gamified 3D reach | Multi-muscle recording from |
| | to grasp task | upper-limb extensors + |
| | | flexors |
| Shoulder Flexion | Gamified 3D reach | Markerless tracking + |
| | to grasp task | Hand device to encourage |
| | | reach to grasp behavior |
| Elbow Extension | Gamified elbow | Markerless tracking |
| | extension task | |
| Pronation/ | Gamified 3D | Markerless tracking + |
| Supination | pointing task | Hand device to encourage |
| | | pronation/supination behavior |

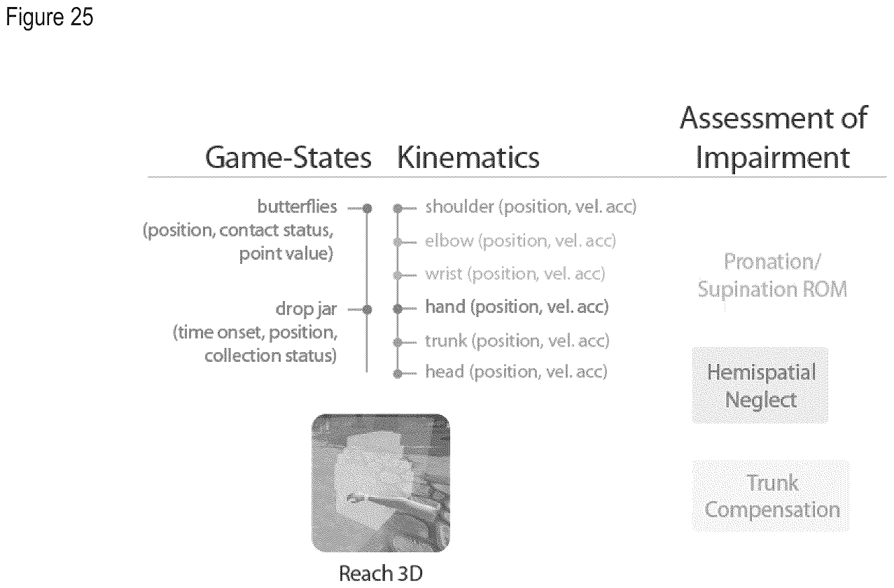

In preferred embodiments, each activity generates a combination of kinematic metrics for all of the body areas used during the activity and game-states data. In some cases, full-body kinematic metrics are generated during an activity, even for areas that are not targeted during the activity. In this way, assessment can be done on secondary effects such as compensation. For some purpose-built activities, this data contains information regarding the impairment features of interest, allowing the system to capture and populate snapshots of a patient's impairment over time during gameplay. FIG. 25 illustrates an exemplary user interface of showing the relationship between game-states, corresponding kinematics for which data is generated and corresponding impairments assessed.

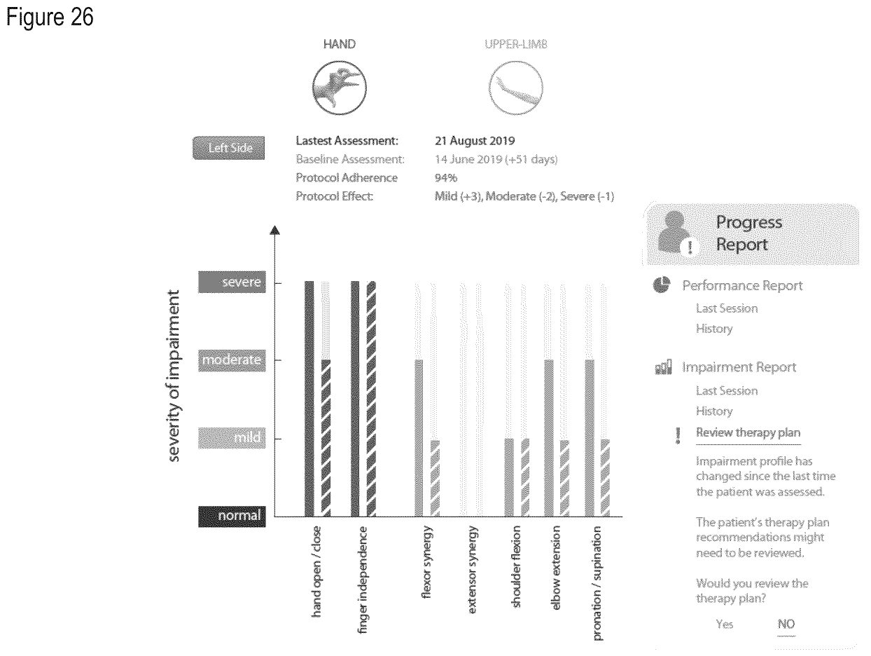

At step 1410, therapy plan efficacy metrics are determined. Preferred embodiments include a repository that provides a snapshot of a patient's performance and impairment during the course of therapy. Since the patient's impairment profile is periodically measured during therapy on a frequent bases through assessments during gamified assessment sessions and therapy activities themselves, the difference between the initial baseline assessment and the current assessment profile can be quantified. For example, in accordance with the exemplary illustration of a user interface in FIG. 26, there has been some improvement in the patient's impairment, specifically impairment in the arm has improved from moderate to mild. Thus, the therapist is prompted to review the therapy plan in order to determine whether the plan needs to be updated in order to account for the impairment change.

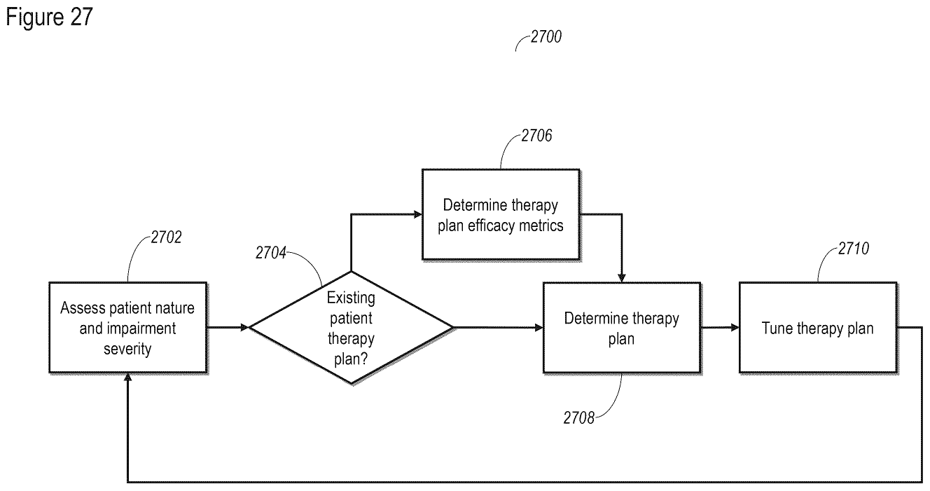

FIG. 27 illustrates an exemplary flowchart of a method 2700 for closed-loop assessment and treatment of a neurological condition. At step 2702, the nature and severity of impairment is assessed. At step 2704, it is determined whether there is an existing therapy plan. If there is, then at step 2706, therapy plan efficacy metrics are determined. At step 2708, a therapy plan is determined. At step 2710, the therapy plan is tuned.

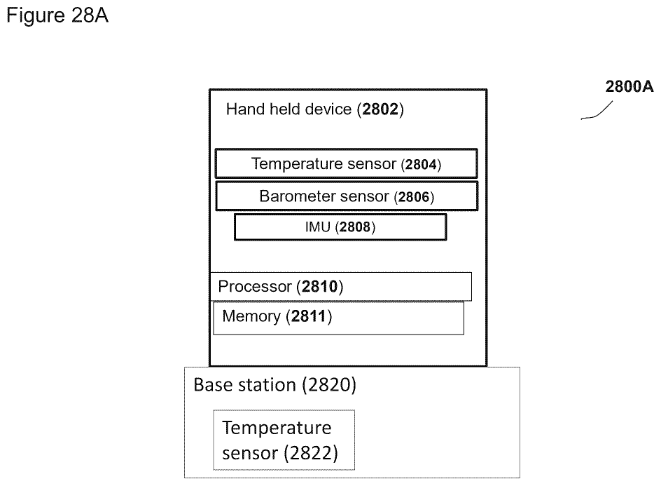

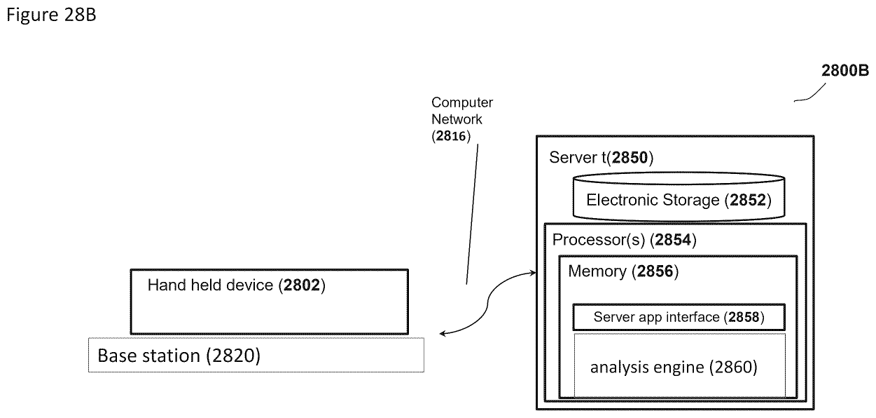

FIGS. 28A and 28B illustrate block diagrams of components of an exemplary hand device in accordance with embodiments. Turning now to FIG. 28A, as shown in a system 2800A, a hand held device 2802 is in electrically conductive communication with a base station 2820 for charging. Hand held device 2802 may be charged directly, for example through insertion of a charging cable (not shown) but preferably is instead charged through base station 2820, such that the charging cable is preferably connected with base station 2820.

Hand held device 2802 comprises a temperature sensor 2802 and a barometric sensor 2806. Hand held device 2802 also optionally and preferably comprises a sensor for obtaining data in regard to movements of hand held device 2802, such as an IMU 2808 as shown. Temperature sensor 2802 provides temperature data in regard to the temperature within hand held device 2802. Barometric sensor 2806 provides barometric (pressure) data in regard to the pressure within hand held device 2802.

Hand held device 2802 also comprises a processor 2810 for executing a plurality of instructions and a memory 2811 for storing such instructions. For example, processor 2810 may execute instructions in regard to the operation of temperature sensor 2802 and barometric sensor 2806. Processor 2810 may also execute instructions in regard to the operation of IMU 2808. Processor 2810 may also execute instructions in regard to preprocessing data from temperature sensor 2802 and barometric sensor 2806, and optionally also from IMU 2808. Processor 2810 may also execute instructions in regard to determining the force applied to hand held device 2802, by analyzing pressure data from barometric sensor 2806 and adjusting such data according to temperature sensor 2802. Alternatively, the force calculation may be determined externally to hand held device 2802, as shown with regard to FIG. 28B. Also alternatively, only pressure and temperature are determined. Optionally temperature correction may be applied according to any suitable algorithm, such as that for example as implemented through the LPS22HH sensor of STMicroelectronics.

Base station 2820 also preferably comprises a temperature sensor 2822 for measuring temperature at base station 2820 as a safety measure.

In operation, hand held device 2802 may be removed from base station 2820 or alternatively remain in contact with base station 2820. Hand held device 2802 is gripped in the hand of a subject. The force placed by the hand of the subject on hand held device 2802 then causes the pressure within hand held device 2802 to increase. Such an increased pressure is measured by barometric sensor 2806. The increased pressure may then be used to estimate applied force. Temperature data from temperature sensor 2804 is then preferably used to refine the calculation of applied force and to increase its accuracy, for example as described in greater detail below. Optionally temperature data from temperature sensor 2804 and pressure data from barometric sensor 2806 are combined to estimate applied force, as applied externally to hand held device 2802, due to manipulation by a hand. Hand held device 2802 preferably comprises, at least in its outer envelope, a flexible material such as silicone for example, or another flexible polymer, to permit deformation of the outer envelope and hence increased internal pressure, from the grip of the hand of the subject that applies force externally to the outer envelope. Preferably such a flexible material is present at a first portion of hand held device 2802, such as a top portion for example and without wishing to be limiting. A second portion of hand held device 2802 preferably comprises a non-flexible material, such as a bottom portion for example and without wishing to be limiting. Optionally and preferably, measurements of applied force, applied by a hand of the subject, are not captured through the second, non-flexible, portion.

Turning now to FIG. 28B, a system 2800B features hand held device 2802 and base station 2820 (shown in simplified form for ease of illustration), in communication with a server 2850 through a computer network 2816, which may be the internet for example. Computer network 2816 may communicate with hand held device 2802, base station 2820, or a combination thereof. Hand held device 2802 may communicate with server 2850 or another computational device through Bluetooth for example. Server 2850 in this embodiment performs the calculations in regard to the determination of force applied externally to hand held device 2802. Server 2850 features an electronic storage 2852, for example for storing additional data and/or models as described herein. Server 2850 also features a processor 2854 for executing instructions stored in a memory 2856. For example, processor 2854 may execute such instructions to combine data from barometric sensor 2806, as well as temperature data from temperature sensor 2804, to calculate force applied to hand held device 2802. If server 2850 is a user computational device with a display screen and user interface, the result of such calculations may then be displayed to the user (not shown).

Server 2850 also features a server app interface 2858 for receiving data from hand held device 2802, base station 2820, or a combination thereof. Such data may then be analyzed by an analysis engine 2860, as operated according to instructions executed by processor 2854. Analysis engine 2860 may be rules based and/or may comprise a trained model, as described in greater detail below.

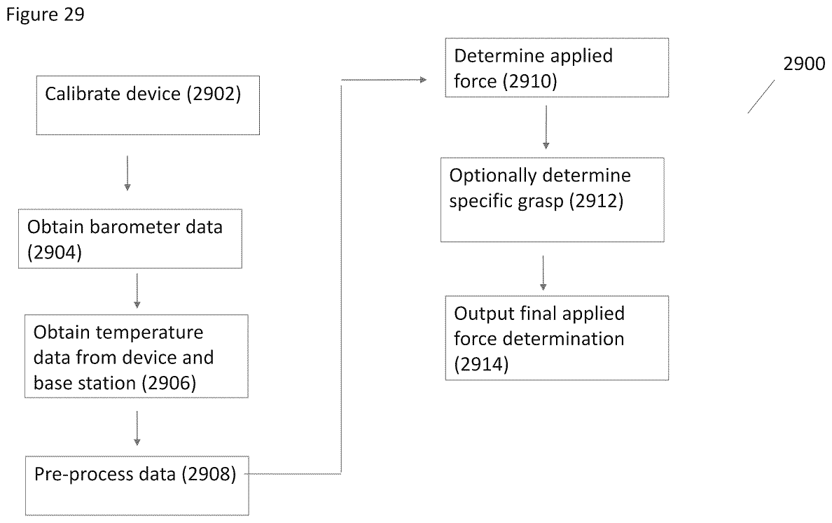

FIG. 29 shows a non-limiting, exemplary method for determining force applied to an exemplary hand device in accordance with embodiments. As shown, a method 2900 begins at 2902, with device calibration. For example, the subject may be asked to grip the hand held device. The subject may also be asked to squeeze the device with minimum force and with maximum force. Calibration in regard to the temperature sensor and the barometric sensor is also performed, preferably while the device is on the base station, more preferably before calibration in regard to activities by the subject begins. Such calibration enables an accurate determination of the internal pressure at the hand held device, as well as an accurate determination of the temperature. Preferably, such calibration features determination of external force applied to the device according to a combination of a reference or standard barometer pressure and temperature, and a measured barometer pressure and temperature as the subject applies external force to (grips) the hand held device.

At 2904, barometer data is obtained from the hand held device as it is gripped by the subject. At 2906, temperature data is preferably obtained from the temperature sensor located within the hand held device. At 2908, the data is preprocessed, whether at the hand held device or externally to the hand held device.

At 2910, force applied to the hand held device is determined from a combination of the data from the barometric sensor and preferably the temperature sensor. As described in greater detail below, the applied force may be determined according to heuristics, through a rules based calculation, and/or according to a trained model. The applied force may be determined at the hand held device or at a remote computational device, such as a server and/or user computational device for example.

At 2912, optionally the specific grasp of the subject is considered in regard to the determination of applied force. For example, the subject may grasp the hand held device with an entire hand, or with a thumb and finger(s), in a pinching grasp. The hand held device may be held by the tips of the thumb and finger(s), or may be held deeper within the palm. These different features of the grasp of the subject may be detected automatically, or may be entered manually by external observation for example. As a non-limiting example, being held deeper within the palm may cause the internal temperature within the hand held device to increase to a greater extent, which may in turn affect the determination of applied force.

At 2914, the final determination of applied external force is output, for example to an external computational device and/or to be displayed to a user.

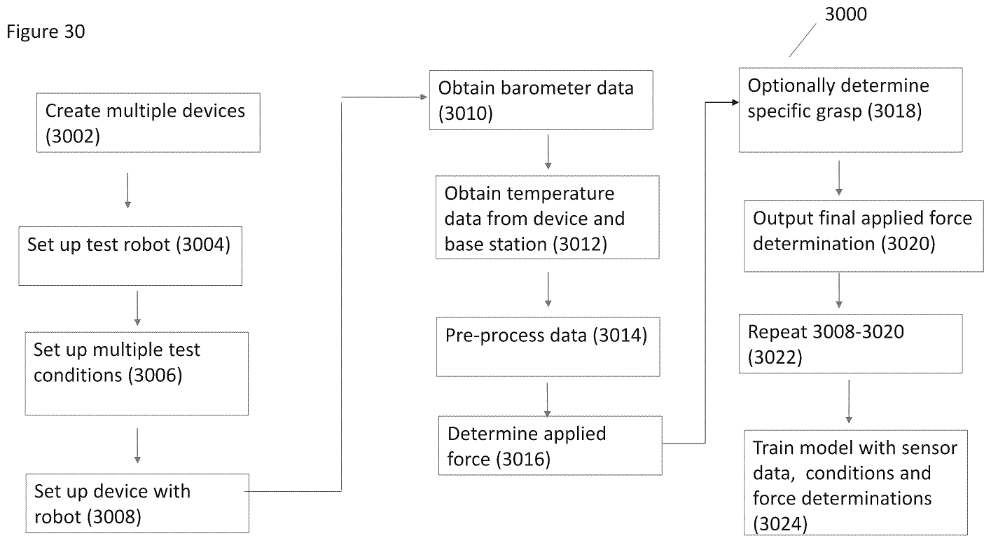

FIG. 30 shows a non-limiting, exemplary method for training a model in regard to determining pressure measurements from an exemplary hand device in accordance with embodiments. As shown in a method 3000, the method preferably begins with creating multiple hand held devices at 3002. Such multiple hand held devices may comprise different external envelope materials and/or may feature other differences, and/or may be constructed according to a single design with a single set of materials. Optionally only one hand held device 3002 is constructed and tested.

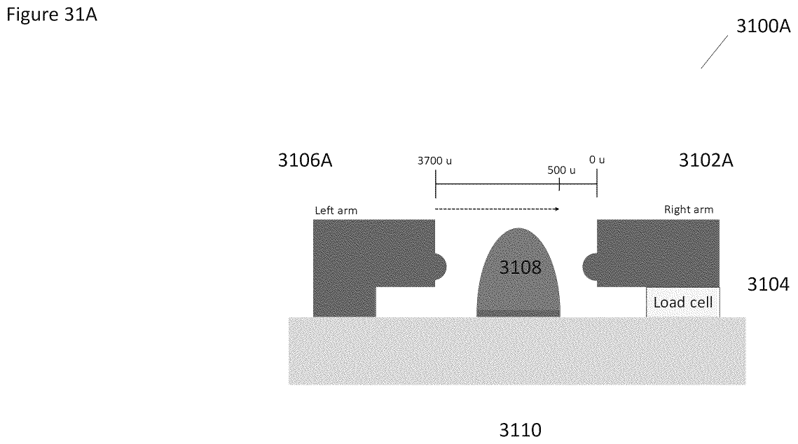

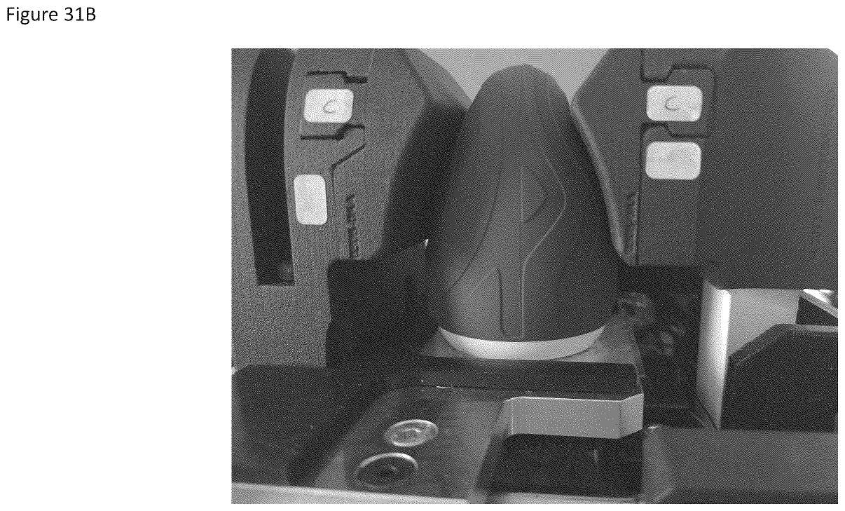

At 3004, a test robot is set up, to be able to provide uniform force on, or “squeezing” of, the hand held device. FIGS. 31A-31B show different exemplary embodiments of the test robot system. As shown with regard to FIG. 31A, a test robot system 3100A features a fixed right arm 3102, in communication with a load cell 3104. A movable left arm 3106 then moves along a linear axis toward right arm 3102. An exemplary hand held device 3108 is “squeezed” between left arm 3106 and right arm 3102. Preferably, items of test robot system 3100A are disposed on a fixed bench 3110. In this example of test robot system 3100A, right arm 3102 and movable left arm 3106 have pinched tips (pinched jaws). FIG. 31B shows a photograph of another such test robot system, in which the moveable left and right jaws now feature an upper projection for grasping. In each of these embodiments, the test robot system is preferably calibrated with a suitable scale.

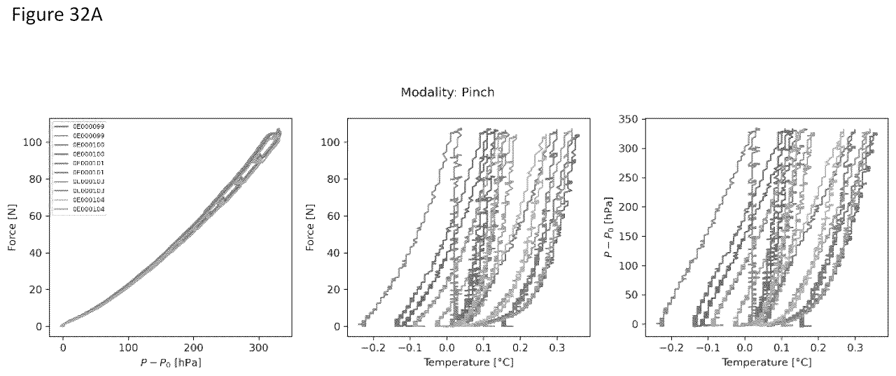

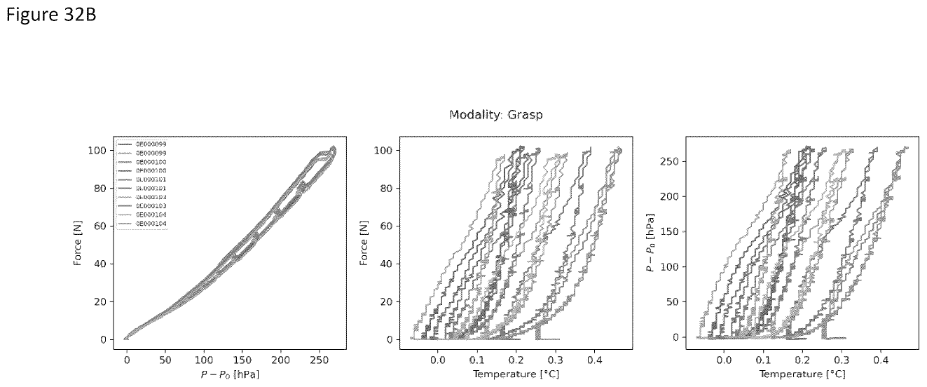

Data reported by the test robot system, shown in FIGS. 32A-32B, indicate a non-linear relationship between the force applied (through the test robot) and the determination of applied external force according to internal sensor data, at lower degrees of applied force. For each of the three graphs in each of FIGS. 32A-32B, the left hand graph shows the relationship between applied force and pressure: the middle graph shows the relationship between applied force and temperature: and the right hand graph shows the relationship between pressure and temperature. All graphs for all three types of test robot systems show a non-linear relationship.

Without wishing to be limited by a single hypothesis, at least a portion of this non-linear relationship may be due to the resistance of the silicone outer envelope, absorbing some of the applied force.

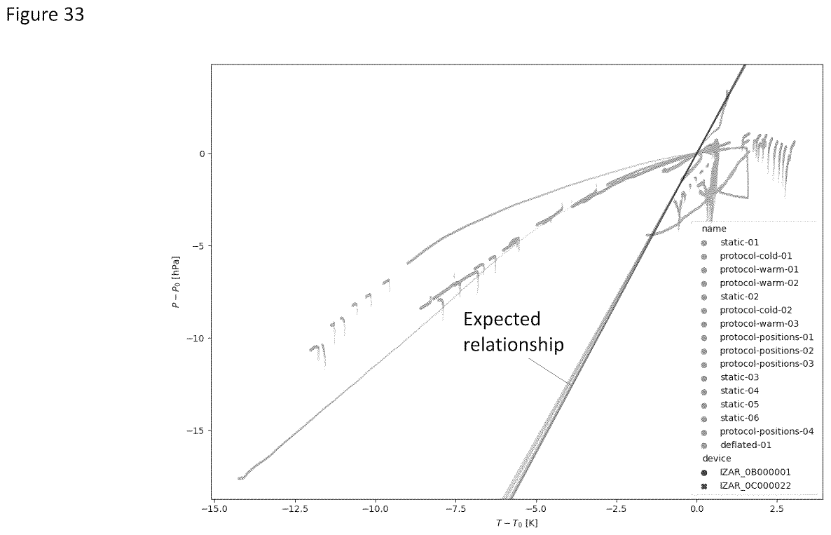

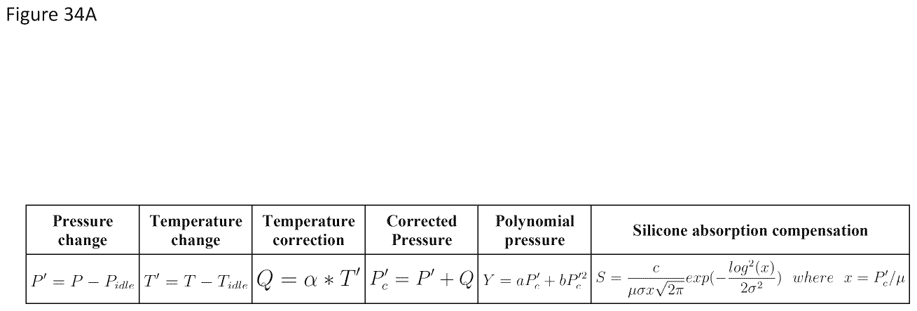

Non-linearity was also observed in terms of the relationship between temperature and pressure, as shown with regard to FIG. 33, for measurements obtained under a variety of temperature and applied force conditions (this data was obtained with a slightly different device). The line labeled “expected relationship” shows the relationship between temperature and pressure that would be expected from the perfect gas law. As shown, the measurements clearly deviate from the expected relationship, indicating a non-linear relationship.

Other observed non-linearities related to the relationship between force and pressure (not shown). Such non-linearity may lead to inaccurate measurement of force application, which may be particularly deleterious when considering the potentially delicate and gradual movements and force applications of the human hand. Such non-linearity may be overcome through the application of a trained model, as described herein.

A trained model is also preferred because the relationship between force and pressure depends only in limited part on the contact surface between the hand held device and the jaws of the test robot (or the finger(s), thumb and/or palm of the subject grasping the hand held device), because the elasticity of the envelope material causes the applied force to be distributed over the envelope. Additional factors, which may contribute to complexity but without wishing to be limited to a closed list, include the mechanical properties of the flexible material of the outer envelope, the shape of the device, the rigid base and the complex, uncontrolled contact surface. The shape of the device is preferably rounded, and may be spherical or non-spherical, but is preferably non-spherical. In this non-limiting example, the hand held device is shown with an ovoid or egg-shape. A non-limiting example of the trained model is described in greater detail below.

Turning back to FIG. 30, at 3006, preferably different test conditions are determined, for example according to the amount of force applied to the hand held device, the temperature, type of jaws (pinched or grasping) and so forth. At 3008, the hand held device is placed in the test robot system, as shown for example in FIGS. 31A and 31B. Barometer data from the barometric sensor within the hand held device is obtained at 3010. Temperature data from within the hand held device is obtained at 3012. At 3014 the data is preprocessed as described herein. The force applied to the hand held device is determined at 3016 as described herein. Optionally, the specific grasp type is determined or considered at 3018, for example according to whether the jaws of the test robot system are flat or pinched. At 3020, the final determination of applied external force is output as described herein.

At 3022, stages 3008 to 3020 are preferably repeated at least once for each hand held device to be tested, and preferably a plurality of times. Also preferably different conditions are tested for each hand held device, for example according to the previously described force gradient for force applied by the test robot system to the hand held device.

At 3024, the model is trained with data related to the temperature and barometric pressure output data, conditions and force determinations.

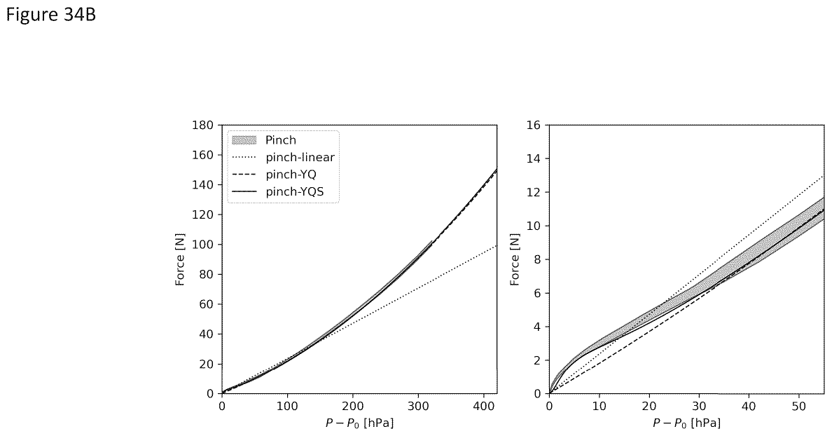

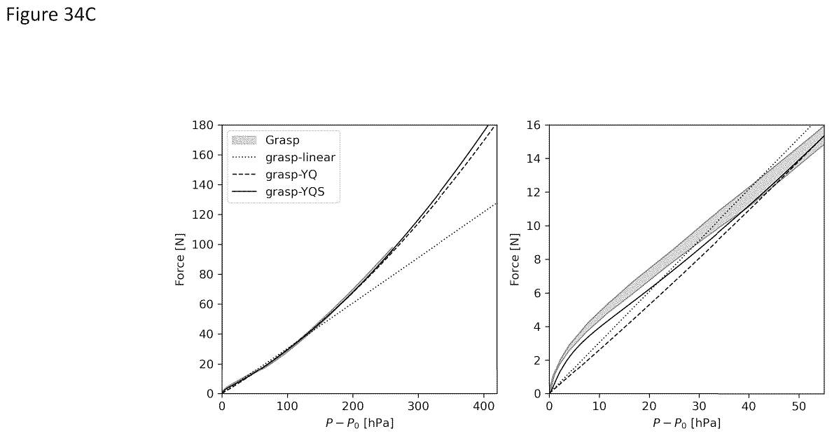

FIG. 34A shows terms that were considered in training the model. The terms indicated with an “idle” subscript relate to the value of that component, such as pressure or temperature, when the device is not being manipulated by a hand. For example, the device may be idle when placed on the base station, and is not being touched or otherwise manipulated by a hand. Testing results show that preferably these values are measured a plurality of times when the device is idle, and more preferably are measured each time that the device is placed on the base station as described herein (results not shown). Optionally, these values are measured after a period of inactivity, such that the device returns to a baseline state. The period of inactivity may be measured for example according to a moving window average of pressure measurements, such that when the pressure measurements fall below a threshold, the timer for the period of inactivity starts. The idle values may then be measured after a sufficiently long period of inactivity has elapsed. Other suitable implementations may be applied for determining baseline measurements.

Without wishing to be limited in any way, various models may be applied, including but not limited to one or more of the following:
A linear model, which may for example feature a linear relationship between a force F and a pressure P applied on area A (“linear”).
A quadratic fit of the curve, along with the temperature correction term Q (“YQ”).
A quadratic fit of the curve, along with the temperature correction term Q. complemented with the S term to compensate for silicone absorption at low forces (“YQS”).

The results of the model fitting are shown in FIGS. 34B (pinch model) and 34C (grasp model).

A non-limiting example of the trained model may be described as follows. Without wishing to be limited, an example of a suitable model features a quadratic fit of the curve, along with the temperature correction term Q, complemented with the S term to compensate for silicone absorption at low forces (YQS). However, according to some embodiments, different models are used for different hand manipulation modalities, such that the YQS model may be used for pinching, while the YQ model may be used for grasping, or vice versa. Optionally the YQ model or the YQS model may be used for both handling modalities. Without wishing to be limited by a single hypothesis, the YQ model may be preferred for overcoming higher temperature sensitivity and greater use of force. While grasping, the whole hand is around the device, transferring more heat, requiring better temperature correction. Forces tend to be higher in grasp than in pinch, meaning that the low forces correction may not be as preferred as for pinching.

The equations for the pinch model may be as follows:

$${P}^{′} = P - {P}_{idle} \qquad ( 3 )$$

$${T}^{′} = T - {T}_{idle} \qquad ( 4 )$$

$$Q = α {T}^{′} \qquad ( 5 )$$

$${P}_{c}^{′} = {P}^{′} + Q \qquad ( 6 )$$

$$Y = {aP}_{c}^{′} + {bP}_{c}^{′ 2} \qquad ( 7 )$$

$$S = \frac{c}{σ {P}_{c}^{′} \sqrt{2 π}} exp ( - \frac{{log}^{2} ( {P}_{c}^{′} / μ )}{2 {σ}^{2}} ) \qquad ( 8 )$$

$$F = Y + S \qquad ( 9 )$$

The equations for the grasp model may be as follows:

$${P}^{′} = P - {P}_{idle} \qquad ( 10 )$$

$${T}^{′} = T - {T}_{idle} \qquad ( 11 )$$

$$Q = α {T}^{′} \qquad ( 12 )$$

$${P}_{c}^{′} = {P}^{′} + Q \qquad ( 13 )$$

$$F = {aP}_{c}^{′} + {bP}_{c}^{′ 2} \qquad ( 14 )$$

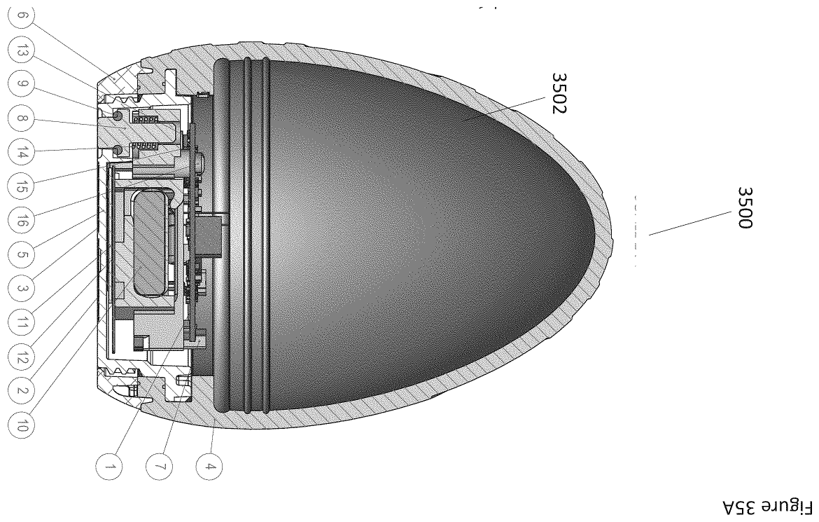

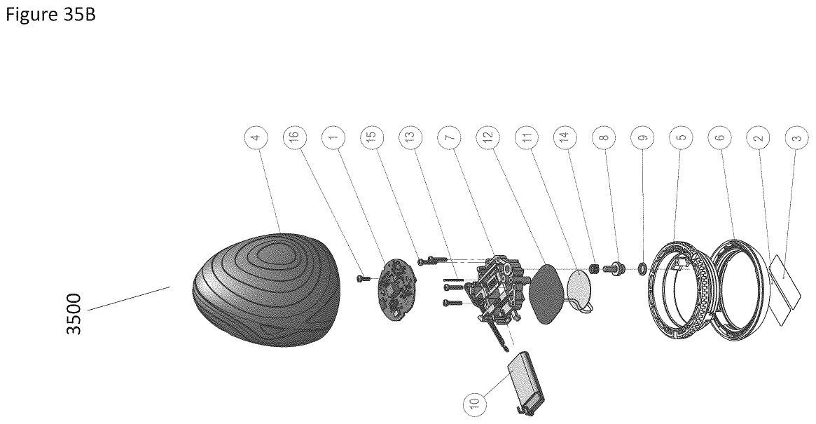

FIG. 35A shows a cross-section of an additional embodiment of the hand-held device. As shown, a hand-held device 3500 features an outer envelope 4, which may comprise silicone or another suitable polymer or other flexible material as described herein. Hand-held device 3500 may feature an ovoid shape as shown herein.

Within outer envelope 4, a chamber 3502 preferably contains air or another gas. A valve 8 controls the flow of air into and from chamber 3502. A spring 14 supports air tightness by applying a force on valve 8, which compresses a seal (shown as O-ring 9) to at least reduce air leakage. Internal pressure can be released through valve 8, thereby enabling internal pressure to be equalized to external air pressure.

A PCB board 1 within chamber 3502 features a plurality of sensors as described herein, including without limitation a temperature sensor, a barometer and a movement sensor, such as an IMU for example. Power may be supplied to PCB board 1 by a battery 10. Preferably battery 10 is rechargeable, more preferably through wireless charging. Coil 11 supports wireless charging. Battery 10 and other electronic components contained within chamber 3502 are preferably protected by ferrite 12.

Screw 15 preferably secures PCB board 1 to an electrobox 7. Electrobox 7 and other components are preferably secured by screws 16 to a base, shown in this non-limiting example as inner ring 5 and outer ring 6. Outer envelope 4 is secured and held between inner ring 5 and outer ring 6.

Electrobox 7 is preferably provided as a structural component, which is the structure on which the electronic components contained within chamber 3502 are mounted. Electrobox 7 preferably also keeps valve 8 under pressure, thereby enabling outer envelope 4 to be maintained in an airtight state.

Light pipe 13 supports debugging, by bringing LED display information from PCB board 1 for external display.

FIG. 35B shows an exploded view of the additional embodiment of the hand-held device of FIG. 35A. The same components have the same references numbers.

Any and all references to publications or other documents, including but not limited to, patents, patent applications, articles, webpages, books, etc., presented in the present application, are herein incorporated by reference in their entirety.

Example embodiments of the devices, systems and methods have been described herein. As noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, systems and apparatuses which may further include any and all elements from any other disclosed methods, systems, and apparatuses, including any and all elements corresponding to target particle separation, focusing/concentration. In other words, elements from one or another disclosed embodiment may be interchangeable with elements from other disclosed embodiments. In addition, one or more features/elements of disclosed embodiments may be removed and still result in patentable subject matter (and thus, resulting in yet more embodiments of the subject disclosure). Correspondingly, some embodiments of the present disclosure may be patentably distinct from one and/or another reference by specifically lacking one or more elements/features. In other words, claims to certain embodiments may contain negative limitation to specifically exclude one or more elements/features resulting in embodiments which are patentably distinct from the prior art which include such features/elements.

What is claimed is:

1. A device for providing dexterous hand function assessment and therapy, comprising:
a first portion of flexible material forming a cavity:
a second portion of non-flexible material:
a plurality of electronics coupled to the second portion: and
wherein a portion of an edge of the first portion is configured to create a partial seal with at least a portion of an edge of the second portion, such that said cavity is partially sealed: said plurality of electronics comprises a pressure sensor, a wireless transceiver, and a power storage unit; said electronics are located within said cavity: and said partial seal is non-hermetic.

2. The device of claim 1, wherein the first portion comprises a graphical component to enable position and orientation tracking of the device.

3. The device of claim 1 or 2, further comprising an active marker to enable position and orientation tracking of the device.

4. The device of claim 3, further comprising a third portion through which the active marker is visible.

5. The device of any of the above claims, wherein said electronics further comprise one or more inertial measurement units (IMU) and a memory storage device.

6. The device of any of the above claims, wherein said pressure sensor comprises a barometric pressure sensor within said cavity for measuring pressure within said cavity.

7. The device of any of the above claims, further comprising a temperature sensor.

8. The device of claim 7, wherein temperature data from said temperature sensor and pressure data from said pressure sensor are transmitted from the device through said wireless transceiver for determination of an external force applied to said first portion of flexible material.

9. The device of any of the above claims, wherein said electronics are anchored to a PCB board.

10. The device of any of the above claims, wherein said first portion of flexible material comprises a material selected from the group consisting of silicone and another flexible polymer.

11. The device of any of the above claims, wherein said second portion of non-flexible material comprises a sufficiently flat facet such that the device can rest upon said facet; and said non-flexible material of said second portion does not deform under the weight of the device when the device is resting on said facet.

12. The device of any of the above claims, wherein said first portion of flexible material is adapted to be manipulated by a hand to receive applied force.

13. The device of claim 12, wherein measurements of said applied force are not captured through said second portion.

14. The device of any of the above claims, wherein said electronics are anchored to said second portion within said cavity, through a stationary and non-deformable anchor point in the second portion.

15. The device of claim 14, wherein said electronics are anchored to said second portion through a PCB.

16. The device of any of the above claims, further comprising a valve to allow the air pressure on the interior of the device to adjust to equilibrium with ambient air pressure.

17. The device of claim 16, wherein said valve is located in said second portion.

18. The device of claim 16, wherein said valve is located in said first portion.

19. The device of any of claims 16-18, further comprising a pressor surface for being pressed for activating said valve.

20. The device of claim 19, further comprising a reset button for resetting said electronics, said reset button comprising said pressor surface.

21. The device of any of claims 16-20, wherein said valve is activated to equalize internal pressure of the device with ambient pressure.

22. The device of any of the above claims, further comprising a reset button for resetting said electronics.

23. The device of any of the above claims, wherein said wireless transceiver uses at least one of a Bluetooth, cellular data, or RFID wireless system.

24. A system for providing dexterous hand function assessment and therapy, comprising:
the device of any of the above claims; and
a base station comprising a battery charging component, the battery charging component including a cooling system.

25. The system of claim 24, further comprising an external computational device:
wherein the pressure within the device is determined according to said pressure sensor data and said temperature sensor data from said temperature sensor within said cavity: and wherein an applied force to the device is determined by said external computational device according to the pressure and the temperature data.

26. The system of claim 25, wherein calibration data is received by said external computational device, said calibration data comprising a first pressure measurement of the interior of the device: wherein said first pressure measurement is a baseline.

27. The system of claim 26, wherein said first pressure measurement comprises a barometric pressure measurement.

28. The system of any of claims 24-27, wherein a calibration process is performed with a user manipulating the device, wherein said calibration process further comprises performing a plurality of manipulations of the device, comprising a movement of the device and a grip on the device, to determine at least one of user range of motion, a maximum pressure for gestures, a minimum acceleration, a maximum acceleration, or a combination thereof.

29. The system of claim 28, wherein said user range of motion is determined from accelerometer data.

30. The system of claim 29, wherein said user range of motion comprises wrist flexion/extension, and wherein a range of motion of wrist flexion/extension is determined by deriving a first plane from an acceleration vector when the device is held by the user at maximum wrist flexion, and by deriving a second plane when the device is held by the user at maximum wrist extension; wherein said range of motion is determined according to an angle between these two vectors on the plane.

31. The system of any of claims 23-30, wherein said wireless transceiver uses at least one of a Bluetooth, cellular data, or RFID wireless system.

32. The system of claim 31, wherein the device comprises a processor for operating firmware, wherein said firmware controls a connection between the device and said external computational device, for streaming data from the device to said external computational device.

33. The system of claim 32, wherein said streaming data is streamed as packets, according to one or more streaming data parameters to select and configure packet content, comprising one or more of timestamp data, acceleration data, gyroscope data, pressure data, temperature data, data normalized according to calibration, or gyroscope derived quaternion data, or a combination thereof.

34. The system of any of the above claims, wherein said accelerometer data is processed through a signal processing to dampen a signal from said accelerometer with minimal impact on latency for use during calibration and activity use.

35. A method for the assessment and therapy of dexterous hand function, comprising:
establishing a connection with a hand device in a system according to any of the above claims:
establishing a hand selection assigned to the hand device:
receiving a first pressure measurement of the interior of the hand device: and
sending an instruction to an idle pressure controller indicating the pressure measurement is a baseline.

36. The method of claim 35, wherein said pressure measurement comprises barometric pressure.

37. The method of claim 35 or 36, further comprising:
receiving temperature data from the hand device; and
determining an external force applied to the hand device at said first portion to at least the first portion according to the temperature data and the pressure data.

38. The method of claim 37, wherein said determining the applied external force is determined according to a trained model.

39. The method of any of the above claims, further comprising performing a calibration process with a user manipulating the device, comprising performing a plurality of manipulations of the device, comprising a movement of the device and a grip on the device, to determine at least one of user range of motion, a maximum pressure for gestures, a minimum acceleration, a maximum acceleration, or a combination thereof.
